(19) United States
(12) Patent Application Publication
(10) Pub. No.: US 2022/0204531 A1
(43) Pub. Date: Jun. 30, 2022

(54) DIBORONIC ACID COMPOUNDS AND METHODS OF MAKING AND USING THEREOF

(71) Applicant: The Regents of The University of California, Oakland, CA (US)

(72) Inventors: Bing Wang, Santa Barbara, CA (US); Kuang-Hua Chou, Santa Barbara, CA (US); Guillermo C. Bazan, Santa Barbara, CA (US); Sumita Pennathur, Santa Barbara, CA (US)

(21) Appl. No.: 17/616,115

(22) PCT Filed: Jun. 4, 2020
(86) PCT No.: PCT/US2020/036167

§ 371 Date: Dec. 2, 2021

(51) Int. Cl. C07F 5/02; A61B 5/145; A61B 5/1477; A61B 5/1455; A61B 5/00; G01N 33/49

(52) U.S. Cl. CPC C07F 5/025; A61B 5/14532; G01N 33/49; A61B 5/1455; A61B 5/685; A61B 5/1477

(57) ABSTRACT

Disclosed are diboronic acid compounds and diboronic acid compound-based sensors for glucose detection, as well as methods for glucose testing in a sample. The diboronic acid compounds allow for selective detection of glucose in the presence of interference sugars, long-term stability, and ease of preparation. Sensors containing the disclosed diboronic acid compounds allow for selective detection of glucose with improved stability at a low cost.

### CROSS-REFERENCE TO RELATED APPLICATIONS

This application claims the benefit of and priority to U.S. Provisional Application No. 62/857,187 filed Jun. 4, 2019, which is hereby incorporated by reference in its entirety.

### FIELD OF THE INVENTION

The invention is generally directed to diboronic acid compounds and methods of making and using thereof, particularly to diboronic acid compounds for analyte detection.

### BACKGROUND OF THE INVENTION

Dysfunction of feedback regulations responsible for controlling glucose levels generally cause diabetes, and can lead to serious complications, such as heart disease, kidney failure, and blindness (Zheng, et al., Nat. Rev. Endocrinol., 14:88-98 (2018); Winocour, Diabetic Med., 35:300-305 (2018); Brownlee, Nature, 414:813-820 (2001)). Continuous glucose monitors (CGMs) are a class of on-body devices that track glucose levels. Most commercially available CGMs employ enzymatic electrochemical glucose-sensing strategies. Due to enzyme instability and drift, these devices suffer from delayed startup times (>two hours), short lifetimes (<two weeks) and require frequent calibration (Rodbard, Diabetes Technol. Ther., 18:3-13 (2016); Chen, et al., Sensors, 17:E182 (2017)). Non-enzymatic catalytic electrochemical sensors have been challenged by selectivity and changes in electrode performance (Rahman, et al., Sensors, 10:4855-4886 (2010); Tian, et al., Mater. Sci. Eng. C Mater. Biol. Appl., 41:100-118 (2014); Dhara, et al., Microchim. Acta, 185:49 (2018)).

Boronic acids (BAs) can form reversible covalent linkages to 1,2- and 1,3-diols, and in particular those present in sugars. In the process of binding diols, BAs become more acidic, with pKa decreases of 2-4 units. The binding-induced change in BA acidity can induce changes in solution pH, electrostatic interactions and surface charge. Although mono boronic acids bind glucose, they also bind to other sugars, such as fructose, galactose and ribose, which are considered interferents in practical glucose-detection platforms.

There remains a need for new compounds that have improved affinity and selectivity for glucose. There is also a need for improved glucose sensors that have improved stability and selectivity for glucose.

Therefore, it is the object of the present invention to provide new compounds that have improved affinity and selectivity for glucose.

It is yet another object of the present invention to provide methods of using such compounds.

It is yet another object of the present invention to provide improved glucose sensors.

### SUMMARY OF THE INVENTION

Diboronic acid compounds with affinity and selectivity for glucose and methods of making and using thereof are disclosed herein. The diboronic acid compounds can have a structure according to Formula I:

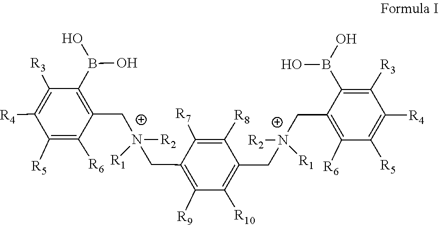

where R₁ and R₂ are independently an unsubstituted alkyl group, a substituted alkyl group, an unsubstituted heteroalkyl group, or a substituted heteroalkyl group; and

where R₃-R₁₀ are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonate ester group containing one substituent, an ether group containing one substituent, an aminooxy group optionally containing one or two substituents at the amino nitrogen, or a hydroxyamino group optionally containing one or two substituents,

wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

Optionally, the diboronic acid compound has a structure according to Formula III:

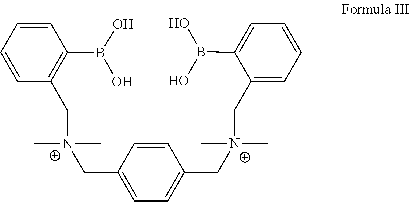 [SMILES: C[N+](C)(Cc1ccc(C[N+](C)(C)Cc2ccccc2B(O)O)cc1)Cc1ccccc1B(O)O]

The diboronic acid compounds described herein optionally include counter ions to the tertiary amine groups. The counter ions can be halide anions, phosphate ion, hydrogen phosphate ion, dihydrogen phosphate ion, trihydrogen phosphate ion, or bicarbonate, or a combination thereof. In some forms, the counter ions are dihydrogen phosphate ions.

Generally, the solubility of the diboronic acid compounds increase with the increase of temperature. The diboronic acid compound can remain aqueous soluble in an aqueous solution at a pH between about 3 and about 11.5.

In some forms, the diboronic acid compound binds glucose with a K_{d} value between about 0.1 and about 30, between about 1 and about 10 mM, between about 2 and about 10 mM, or between about 2 mM and about 5 mM.

In some forms, the diboronic acid compound binds glucose with a K_{d} value at least about 2-times lower, preferably at least about 15-times lower, more preferably at least about 20-times lower than a K_{d} value for an interference sugar under the same conditions. Typical interference sugars include fructose, galactose, maltose, sucrose, lactose, or a combination thereof.

In some forms, the diboronic acid compounds have a pKa value between about 7.4 and about 10.5, preferably between about 8.5 and about 10.5, more preferably between about 9 and about 10. Generally, the pKa value of the diboronic acid compounds decreases upon binding with glucose. The pKa value of the diboronic acid compounds may increase or decrease by about 1 unit, about 2 units, preferably about 3 units, optionally by about 4 units upon binding with glucose. Typically, the pKa value of the diboronic acid compounds decreases upon binding with glucose. For example, the pKa value of the diboronic acid compounds decreases by about 1 unit, about 2 units, preferably about 3 units, optionally by about 4 units upon binding with glucose. In a particular form, the pKa value of the diboronic acid compounds decreases from about 9.4 to about 6.3 upon binding with glucose.

A conductivity sensor for measuring glucose concentration in a biological sample including one or more of the diboronic acid compounds is disclosed. The conductivity sensor includes a reservoir containing the diboronic acid compound(s) and a buffer solution or buffer salts, a pair of electrodes, a membrane, and optionally a detector. The electrodes are in electrical communication with each other. When a buffer solution is present, the diboronic acid compound(s) are in the buffer solution and an electrically conductive surface of each electrode is in contact with the buffer solution. The membrane is configured to prevent or reduce ion exchange between the buffer solution and the biological sample.

When buffer salts are present in the reservoir, the diboronic acid compound(s) and buffer salt(s) in a solid form, optionally in the form of a powder, film, or tablet. In these conductivity sensors, a solvent, such as water or an aqueous solvent, is added to dissolve the diboronic acid compound(s) and buffer salt(s) to form a buffer solution prior to using the sensor.

The sample reservoir is typically defined by side walls and a bottom surface, and contains an opening configured to allow the biological sample to enter the reservoir. At least a portion of the bottom surface and/or one or both of the side walls of the reservoir is formed from the electrically conductive surface of each of the electrodes. Optionally, the electrically conductive surfaces of the electrodes are located on and form part of the bottom surface of the reservoir.

The membrane is located adjacent to the opening of the reservoir, and defines an outer surface that encloses the buffer solution or solid buffer salts and diboronic acid compound inside of the reservoir.

Methods of testing the presence, the absence, or the concentration of glucose in a biological sample (e.g. blood containing an unknown concentration of glucose) using the conductivity sensor are also disclosed. The method includes: (a) applying a voltage at a frequency; (b) measuring a first resistance of the buffer solution; (c) transferring the biological sample to the buffer solution to form a test sample; and (d) measuring a second resistance of the test sample. In some forms, the second resistance is lower than the first resistance. In some forms, the difference between the first resistance and the second resistance is a function of glucose concentration. Optionally, steps (c) and (d) are repeated two or more times. Typically, the sensors can detect glucose from 0 to about 30 mM, from about 5 mM to about 20 mM, from about 12 mM to about 30 mM, or from about 2 mM to about 30 mM. In some forms, the difference between the first resistance and the second resistance in response to an interference sugar is less than about 3% as compared to the difference between the first resistance and the second resistance in response to glucose. In some forms, the voltage is between about 1 mV and about 20 mV, preferably about 20 mV. In some forms, the frequency is between about 1 kHz and about 1 MHz, preferably about 10⁵ Hz.

An optical sensor including the diboronic acid compounds is disclosed. Typically, the optical sensor includes a dye, a light source, and a detector, where the diboronic acid compound and the dye form a complex (DBA-D complex).

Methods of testing the presence, the absence, or the concentration of glucose in a biological sample using the optical sensor are also disclosed. The method includes: (a) measuring a first optical signal (such as absorbance or fluorescence) of the DBA-D complex; (b) adding the biological sample to the optical sensor such that the biological sample is in contact with the DBA-D complex; and (c) measuring a second optical signal (such as absorbance or fluorescence) of the DBA-D complex. The optical signal of the DBA-D complex increases or decreases upon the addition of the sample as a function of glucose concentration. Optionally, steps (b) and (c) are repeated two or more times.

Exemplary continuous glucose monitoring sensors are disclosed. The continuous glucose monitoring sensor includes: (a) a conductivity sensor or an optical sensor described above; and optionally (b) a bipolar membrane; and/or (c) a microneedle, optionally an array of microneedles for fluid extraction.

Also disclosed is an exemplary continuous glucose monitoring sensing patch, which includes two or more of the above-described continuous glucose monitoring sensors.

### BRIEF DESCRIPTION OF THE DRAWINGS

FIG. 1 is a graph showing glucose-dependent pKa of DBA2+ by measuring absorbance at 280 nm and fitting the curve as a function of pH. Absorbance of 100 μL of 1 mM DBA2+Br as a function of pH in the absence (square) or presence (star) of 200 mM glucose with calculated pKa indicated.

FIG. 2 is a graph showing absorbance of 200 μL of 1 mM DBA2+Br at 280 nm in 50 mM phosphate buffer at pH 7.4 in the presence of various concentrations of glucose and other sugars as indicated. Dissociation constants were determined by fitting curves as a function of sugar concentration.

FIG. 3 is a schematic of the device used in the Examples for impedance spectra and time resolution monitoring at high frequency.

FIG. 4A is a graph showing the electrical impedance spectra of the testing solution at the frequency from 10 Hz to 10 MHz. FIG. 4B is graph showing a magnified view of the region indicated by an arrow in FIG. 4A. FIG. 4C is a graph showing the impedance phase curve as a function of scanning frequency. The area pointed by the arrow shows negligible reactance compared to resistance.

FIG. 5 is an illustration of the binding of DBA2+ to glucose in PBS buffer solution, showing changes to solution conductivity due to the difference in the composition of ions.

FIG. 6A is a graph showing the solution resistance (R) of 1 mL of test solution changes with continues addition of 0.5 M or 2 M glucose concentration. After adding glucose to 30 mM, the testing solution was diluted to 12 mM (star) to confirm repeatability. FIG. 6B is a graph showing the R of 1 mL of test solution changes with continues addition of water at the same volume as that in glucose addition experiment as a control.

FIG. 7A is a graph showing changes in solution resistance (R, left, black) or conductance (6, right, grey) as a function of glucose concentration (n=4 independent experiments at RT). Values are expressed as a percentage, normalized to the initial resistance (R₀) and conductance (δ₀) of the testing solution. FIG. 7B is a graph showing changes in R upon addition of low (5 mM) or high (20 mM) glucose solutions (Glu), followed by addition of 1 mM fructose (Fru) or galactose (Gal).

FIG. 8A is an illustration of the competitive binding between glucose with DBA2+ and Alizarin Red S (ARS) with DBA2+ in buffer and the dual mode detection of florescence and absorbance (i.e. transmission) signals that allows self-calibration using algorithms. FIG. 8B is a graph showing the change of absorption spectra of the ARS/DBA2+ complex with the increase of glucose concentrations. FIG. 8C is a graph showing the decrease in fluorescence of the ARS/DBA2+ complex with the increase of glucose concentrations.

FIG. 9A is a schematic of an exemplary continuous glucose monitoring system (CGMS) in the form of a patch which contains a plurality of continuous glucose monitoring sensors and an array of hollow microneedles. FIG. 9B is a magnified, exploded view of one continuous glucose monitoring sensor.

FIG. 10A is a graph showing calibration curves generated by plotting absorbance values vs. fluorescence values measured in standard glucose solutions containing ARS and DBA2+ at varied concentrations. FIG. 10B is a graph showing the calculation curves generated by plotting absorbance or fluorescence values vs. glucose concentrations measured in standard glucose solutions containing ARS and DBA2+ at fixed concentrations.

FIG. 11 is a schematic of an exemplary optical sensor.

### DETAILED DESCRIPTION OF THE INVENTION

### I. Definitions

As used herein, the term “alkyl” refers to univalent groups derived from alkanes by removal of a hydrogen atom from any carbon atom. Alkanes represent saturated hydrocarbons, including those that are cyclic (either monocyclic or polycyclic). Alkyl groups can be linear, branched, or cyclic. Suitable alkyl groups have one to 30 carbon atoms, i.e., C₁-C₃₀ alkyl. If the alkyl is branched, it is understood that at least four carbons are present. If the alkyl is cyclic, it is understood that at least three carbons are present.

As used herein, the term “heteroalkyl” refers to alkyl groups where one or more carbon atoms are replaced with a heteroatom, such as, O, N, or S. Heteroalkyl groups can be linear, branched, or cyclic. Suitable heteroalkyl groups have one to 30 carbon atoms, i.e., C₁-C₃₀ heteroalkyl. If the heteroalkyl is branched, it is understood that at least four carbons are present. If the heteroalkyl is cyclic, it is understood that at least two carbons and at least one heteroatom are present.

As used herein, the term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear, branched, or cyclic. Suitable alkenyl groups have two to 30 carbon atoms, i.e., C₂-C₃₀ alkenyl. If the alkenyl is branched, it is understood that at least four carbons are present. If the alkenyl is cyclic, it is understood that at least three carbons are present.

As used herein, the term “heteroalkenyl” refers to alkenyl groups in which one or more doubly bonded carbon atoms are replaced by a heteroatom. Heteroalkenyl groups can be linear, branched, or cyclic. Suitable heteroalkenyl groups have two to 30 carbon atoms, i.e., C₂-C₃₀ heteroalkenyl. If the heteroalkenyl is branched, it is understood that at least four carbons are present. If heteroalkenyl is cyclic, it is understood that at least two carbons and at least one heteroatom are present.

As used herein, the term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear, branched, or cyclic. Suitable alkynyl groups have two to 30 carbon atoms, i.e., C₂-C₃₀ alkynyl. If the alkynyl is branched, it is understood that at least four carbons are present. If alkynyl is cyclic, it is understood that at least three carbons are present.

As used herein, the term “heteroalkynyl” refers to alkynyl groups in which one or more triply bonded carbon atoms are replaced by a heteroatom. Heteroalkynyl groups can be linear, branched, or cyclic. Suitable heteroalkynyl groups have two to 30 carbon atoms, i.e., C₂-C₃₀ heteroalkynyl. If the heteroalkynyl is branched, it is understood that at least four carbons are present. If heteroalkynyl is cyclic, it is understood that at least two carbons and at least one heteroatom are present.

As used herein, the term “aryl” refers to univalent groups derived from arenes by removal of a hydrogen atom from a ring atom. Arenes are monocyclic and polycyclic aromatic hydrocarbons. In polycyclic aryl groups, the rings can be attached together in a pendant manner or can be fused. Suitable aryl groups have six to 50 carbon atoms, i.e., C₆-C₅₀ aryl.

As used herein, the term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hickel rule (4n+2). In polycyclic heteroaryl groups, the rings can be attached together in a pendant manner or can be fused. Suitable heteroaryl groups have three to 50 carbon atoms, i.e., C₃-C₅₀ heteroaryl.

As used herein, the term “interference sugar” refers to a sugar other than glucose, such as fructose, galactose, maltose, sucrose, or lactose, that is present in the body of a subject.

Numerical ranges disclosed in the present application of any type, disclose individually each possible number that such a range could reasonably encompass, as well as any sub-ranges and combinations of sub-ranges encompassed therein.

### II. Diboronic Acid Compound

Disclosed herein are diboronic acid compounds. The diboronic acid compounds are soluble in aqueous solutions. Typically, the diboronic acid compound has a solubility of at least about 1 g/L in an aqueous solution at pH about 7.4, temperature about 25° C. The diboronic acid compounds can remain soluble in an aqueous solution over a pH range from about 3 to about 11.5. Optionally, prior to binding with glucose, the diboronic acid compounds have a solubility between about 1 g/L and about 5 g/L, between about 1.5 g/L and about 5 g/L, between about 2 g/L and about 5 g/L, between about 2.5 g/L and about 5 g/L, between about 1 g/L and about 4.5 g/L, or between about 1 g/L and about 4 g/L.

Following binding with glucose, the solubility of the diboronic acid compounds typically increases. Generally, upon binding with glucose, the diboronic acid compounds have a solubility >5 g/L in an aqueous solution at a pH a pH between about 3 and about 11.5, such as at a pH of about 7.4, and 25° C. Optionally, the diboronic acid compounds have a solubility between about 5 g/L and about 36 g/L, between about 5 g/L and about 30 g/L, between about 5 g/L and about 25 g/L, between about 5 g/L and about 20 g/L, between about 5 g/L and about 15 g/L, or between about 5 g/L and about 10 g/L, in an aqueous solution at a pH between about 3 and about 11.5, such as at a pH of about 7.4, and 25° C.

The diboronic acid compounds have high affinity and selectivity to glucose. For example, generally the dissociation constant for the affinity of the diboronic acids to glucose is lower than about 1.5 mM. Additionally, generally the dissociation constant for the diboronic acids to glucose is generally at least about 2-times lower than the dissociation constant for the diboronic acids bound to an interference sugars, such as fructose, galactose, maltose, sucrose, or lactose, under the same conditions (e.g. the same temperature, pressure, solution, pH, etc.). Further the pKa for the diboronic acids changes following the binding of glucose to the diboronic acids.

The diboronic acid compounds can be described as having a structure of Formula I or a salt thereof:

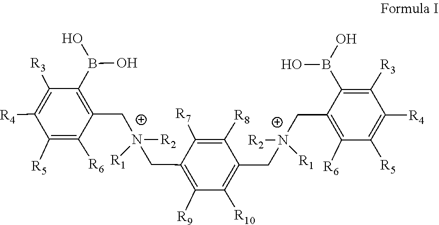

where R₁ and R₂ are independently an unsubstituted alkyl group, a substituted alkyl group, an unsubstituted heteroalkyl group, or a substituted heteroalkyl group; and

where R₃-R₁₀ are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group; a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

The alkyl group can be linear, branched, or cyclic. A C₁-C₃₀ alkyl can be a linear C₁-C₃₀ alkyl, a branched C₁-C₃₀ alkyl, a cyclic C₁-C₃₀ alkyl, a linear or branched C₁-C₃₀ alkyl, a linear or cyclic C₁-C₃₀ alkyl, a branched or cyclic C₁-C₃₀ alkyl, or a linear, branched, or cyclic C₁-C₃₀ alkyl. Optionally, alkyl groups have one to 20 carbon atoms, i.e., C₁-C₂₀ alkyl. In some forms, a C₁-C₂₀ alkyl can be a linear C₁-C₂₀ alkyl, a branched C₁-C₂₀ alkyl, a cyclic C₁-C₂₀ alkyl, a linear or branched C₁-C₂₀ alkyl, a branched or cyclic C₁-C₂₀ alkyl, or a linear, branched, or cyclic C₁-C₂₀ alkyl. Optionally, alkyl groups have one to 10 carbon atoms, i.e., C₁-C₁₀ alkyl. In some forms, a C₁-C₁₀ alkyl can be a linear C₁-C₁₀ alkyl, a branched C₁-C₁₀ alkyl, a cyclic C₁-C₁₀ alkyl, a linear or branched C₁-C₁₀ alkyl, a branched or cyclic C₁-C₁₀ alkyl, or a linear, branched, or cyclic C₁-C₁₀ alkyl. Optionally, alkyl groups have one to 6 carbon atoms, i.e., C₁-C₆ alkyl. In some forms, a C₁-C₆ alkyl can be a linear C₁-C₆ alkyl, a branched C₁-C₆ alkyl, a cyclic C₁-C₆ alkyl, a linear or branched C₁-C₆ alkyl, a branched or cyclic C₁-C₆ alkyl, or a linear, branched, or cyclic C₁-C₆ alkyl. Optionally, alkyl groups have one to four carbons, i.e., C₁-C₄ alkyl. In some forms, a C₁-C₄ alkyl can be a linear C₁-C₄ alkyl, a branched C₁-C₄ alkyl, a cyclic C₁-C₄ alkyl, a linear or branched C₁-C₄ alkyl, a branched or cyclic C₁-C₄ alkyl, or a linear, branched, or cyclic C₁-C₄ alkyl.

The heteroalkyl group can be linear, branched, or cyclic. A C₁-C₃₀ heteroalkyl can be a linear C₁-C₃₀ heteroalkyl, a branched C₁-C₃₀ heteroalkyl, a cyclic C₁-C₃₀ heteroalkyl, a linear or branched C₁-C₃₀ heteroalkyl, a linear or cyclic C₁-C₃₀ heteroalkyl, a branched or cyclic C₁-C₃₀ heteroalkyl, or a linear, branched, or cyclic C₁-C₃₀ heteroalkyl. Optionally, heteroalkyl groups have one to 20 carbon atoms, i.e., C₁-C₂₀ heteroalkyl. In some forms, a C₁-C₂₀ heteroalkyl can be a linear C₁-C₂₀ heteroalkyl, a branched C₁-C₂₀ heteroalkyl, a cyclic C₁-C₂₀ heteroalkyl, a linear or branched C₁-C₂₀ heteroalkyl, a branched or cyclic C₁-C₂₀ heteroalkyl, or a linear, branched, or cyclic C₁-C₂₀ heteroalkyl. Optionally, heteroalkyl groups have one to 10 carbon atoms, i.e., C₁-C₁₀ heteroalkyl. In some forms, a C₁-C₁₀ heteroalkyl can be a linear C₁-C₁₀ heteroalkyl, a branched C₁-C₁₀ heteroalkyl, a cyclic C₁-C₁₀ heteroalkyl, a linear or branched C₁-C₁₀ heteroalkyl, a branched or cyclic C₁-C₁₀ heteroalkyl, or a linear, branched, or cyclic C₁-C₁₀ heteroalkyl. Optionally, heteroalkyl groups have one to 6 carbon atoms, i.e., C₁-C₆ heteroalkyl. In some forms, a C₁-C₆ heteroalkyl can be a linear C₁-C₆ heteroalkyl, a branched C₁-C₆ heteroalkyl, a cyclic C₁-C₆ heteroalkyl, a linear or branched C₁-C₆ heteroalkyl, a branched or cyclic C₁-C₆ heteroalkyl, or a linear, branched, or cyclic C₁-C₆ heteroalkyl. Optionally, heteroalkyl groups have one to four carbons, i.e., C₁-C₄ heteroalkyl. In some forms, a C₁-C₄ heteroalkyl can be a linear C₁-C₄ heteroalkyl, a branched C₁-C₄ heteroalkyl, a cyclic C₁-C₄ heteroalkyl, a linear or branched C₁-C₄ heteroalkyl, a branched or cyclic C₁-C₄ heteroalkyl, or a linear, branched, or cyclic C₁-C₄ heteroalkyl.

The alkenyl group can be linear, branched, or cyclic. A C₂-C₃₀ alkenyl can be a linear C₂-C₃₀ alkenyl, a branched C₂-C₃₀ alkenyl, a cyclic C₂-C₃₀ alkenyl, a linear or branched C₂-C₃₀ alkenyl, a linear or cyclic C₂-C₃₀ alkenyl, a branched or cyclic C₂-C₃₀ alkenyl, or a linear, branched, or cyclic C₂-C₃₀ alkenyl. Optionally, alkenyl groups have two to 20 carbon atoms, i.e., C₂-C₂₀ alkenyl. In some forms, a C₂-C₂₀ alkenyl can be a linear C₂-C₂₀ alkenyl, a branched C₂-C₂₀ alkenyl, a cyclic C₂-C₂₀ alkenyl, a linear or branched C₂-C₂₀ alkenyl, a branched or cyclic C₂-C₂₀ alkenyl, or a linear, branched, or cyclic C₂-C₂₀ alkenyl. Optionally, alkenyl groups have two to 10 carbon atoms, i.e., C₂-C₁₀ alkenyl. In some forms, a C₂-C₁₀ alkenyl can be a linear C₂-C₁₀ alkenyl, a branched C₂-C₁₀ alkenyl, a cyclic C₂-C₁₀ alkenyl, a linear or branched C₂-C₁₀ alkenyl, a branched or cyclic C₂-C₁₀ alkenyl, or a linear, branched, or cyclic C₂-C₂₀ alkenyl. Optionally, alkenyl groups have two to 6 carbon atoms, i.e., C₂-C₆ alkenyl. In some forms, a C₂-C₆ alkenyl can be a linear C₂-C₆ alkenyl, a branched C₂-C₆ alkenyl, a cyclic C₂-C₆ alkenyl, a linear or branched C₂-C₆ alkenyl, a branched or cyclic C₂-C₆ alkenyl, or a linear, branched, or cyclic C₂-C₆ alkenyl. Optionally, alkenyl groups have two to four carbons, i.e., C₂-C₄ alkenyl. In some forms, a C₂-C₄ alkenyl can be a linear C₂-C₄ alkenyl, a branched C₂-C₄ alkenyl, a cyclic C₂-C₄ alkenyl, a linear or branched C₂-C₄ alkenyl, a branched or cyclic C₂-C₄ alkenyl, or a linear, branched, or cyclic C₂-C₄ alkenyl.

The heteroalkenyl group can be linear, branched, or cyclic. A C₂-C₃₀ heteroalkenyl can be a linear C₂-C₃₀ heteroalkenyl, a branched C₂-C₃₀ heteroalkenyl, a cyclic C₂-C₃₀ heteroalkenyl, a linear or branched C₂-C₃₀ heteroalkenyl, a linear or cyclic C₂-C₃₀ heteroalkenyl, a branched or cyclic C₂-C₃₀ heteroalkenyl, or a linear, branched, or cyclic C₂-C₃₀ heteroalkenyl. Optionally, heteroalkenyl groups have two to 20 carbon atoms, i.e., C₂-C₂₀ heteroalkenyl. In some forms, a C₂-C₂₀ heteroalkenyl can be a linear C₂-C₂₀ heteroalkenyl, a branched C₂-C₂₀ heteroalkenyl, a cyclic C₂-C₂₀ heteroalkenyl, a linear or branched C₂-C₂₀ heteroalkenyl, a branched or cyclic C₂-C₂₀ heteroalkenyl, or a linear, branched, or cyclic C₂-C₂₀ heteroalkenyl. Optionally, heteroalkenyl groups have two to 10 carbon atoms, i.e., C₂-C₁₀ heteroalkenyl. In some forms, a C₂-C₁₀ heteroalkenyl can be a linear C₂-C₁₀ heteroalkenyl, a branched C₂-C₁₀ heteroalkenyl, a cyclic C₂-C₁₀ heteroalkenyl, a linear or branched C₂-C₁₀ heteroalkenyl, a branched or cyclic C₂-C₁₀ heteroalkenyl, or a linear, branched, or cyclic C₂-C₂₀ heteroalkenyl. Optionally, heteroalkenyl groups have two to 6 carbon atoms, i.e., C₂-C₆ heteroalkenyl. In some forms, a C₂-C₆ heteroalkenyl can be a linear C₂-C₆ heteroalkenyl, a branched C₂-C₆ heteroalkenyl, a cyclic C₂-C₆ heteroalkenyl, a linear or branched C₂-C₆ heteroalkenyl, a branched or cyclic C₂-C₆ heteroalkenyl, or a linear, branched, or cyclic C₂-C₆ heteroalkenyl. Optionally, heteroalkenyl groups have two to four carbons, i.e., C₂-C₄ heteroalkenyl. In some forms, a C₂-C₄ heteroalkenyl can be a linear C₂-C₄ heteroalkenyl, a branched C₂-C₄ heteroalkenyl, a cyclic C₂-C₄ heteroalkenyl, a linear or branched C₂-C₄ heteroalkenyl, a branched or cyclic C₂-C₄ heteroalkenyl, or a linear, branched, or cyclic C₂-C₄ heteroalkenyl.

The alkynyl group can be linear, branched, or cyclic. A C₂-C₃₀ alkynyl can be a linear C₂-C₃₀ alkynyl, a branched C₂-C₃₀ alkynyl, a cyclic C₂-C₃₀ alkynyl, a linear or branched C₂-C₃₀ alkynyl, a linear or cyclic C₂-C₃₀ alkynyl, a branched or cyclic C₂-C₃₀ alkynyl, or a linear, branched, or cyclic C₂-C₃₀ alkynyl. Optionally, alkynyl groups have two to 20 carbon atoms, i.e., C₂-C₂₀ alkynyl. In some forms, a C₂-C₂₀ alkynyl can be a linear C₂-C₂₀ alkynyl, a branched C₂-C₂₀ alkynyl, a cyclic C₂-C₂₀ alkynyl, a linear or branched C₂-C₂₀ alkynyl, a branched or cyclic C₂-C₂₀ alkynyl, or a linear, branched, or cyclic C₂-C₂₀ alkynyl. Optionally, alkynyl groups have two to 10 carbon atoms, i.e., C₂-C₁₀ alkynyl. In some forms, a C₂-C₁₀ alkynyl can be a linear C₂-C₁₀ alkynyl, a branched C₂-C₁₀ alkynyl, a cyclic C₂-C₁₀ alkynyl, a linear or branched C₂-C₁₀ alkynyl, a branched or cyclic C₂-C₁₀ alkynyl, or a linear, branched, or cyclic C₂-C₂₀ alkynyl. Optionally, alkynyl groups have two to 6 carbon atoms, i.e., C₂-C₆ alkynyl. In some forms, a C₂-C₆ alkynyl can be a linear C₂-C₆ alkynyl, a branched C₂-C₆ alkynyl, a cyclic C₂-C₆ alkynyl, a linear or branched C₂-C₆ alkynyl, a branched or cyclic C₂-C₆ alkynyl, or a linear, branched, or cyclic C₂-C₆ alkynyl. Optionally, alkynyl groups have two to four carbons, i.e., C₂-C₄ alkynyl. In some forms, a C₂-C₄ alkynyl can be a linear C₂-C₄ alkynyl, a branched C₂-C₄ alkynyl, a cyclic C₂-C₄ alkynyl, a linear or branched C₂-C₄ alkynyl, a branched or cyclic C₂-C₄ alkynyl, or a linear, branched, or cyclic C₂-C₄ alkynyl.

The heteroalkynyl group can be linear, branched, or cyclic. A C₂-C₃₀ heteroalkynyl can be a linear C₂-C₃₀ heteroalkynyl, a branched C₂-C₃₀ heteroalkynyl, a cyclic C₂-C₃₀ heteroalkynyl, a linear or branched C₂-C₃₀ heteroalkynyl, a linear or cyclic C₂-C₃₀ heteroalkynyl, a branched or cyclic C₂-C₃₀ heteroalkynyl, or a linear, branched, or cyclic C₂-C₃₀ heteroalkynyl. Optionally, heteroalkynyl groups have two to 20 carbon atoms, i.e., C₂-C₂₀ heteroalkynyl. In some forms, a C₂-C₂₀ heteroalkynyl can be a linear C₂-C₂₀ heteroalkynyl, a branched C₂-C₂₀ heteroalkynyl, a cyclic C₂-C₂₀ heteroalkynyl, a linear or branched C₂-C₂₀ heteroalkynyl, a branched or cyclic C₂-C₂₀ heteroalkynyl, or a linear, branched, or cyclic C₂-C₂₀ heteroalkynyl. Optionally, heteroalkynyl groups have two to 10 carbon atoms, i.e., C₂-C₁₀ heteroalkynyl. In some forms, a C₂-C₁₀ heteroalkynyl can be a linear C₂-C₁₀ heteroalkynyl, a branched C₂-C₁₀ heteroalkynyl, a cyclic C₂-C₁₀ heteroalkynyl, a linear or branched C₂-C₁₀ heteroalkynyl, a branched or cyclic C₂-C₁₀ heteroalkynyl, or a linear, branched, or cyclic C₂-C₂₀ heteroalkynyl. Optionally, heteroalkynyl groups have two to 6 carbon atoms, i.e., C₂-C₆ heteroalkynyl. In some forms, a C₂-C₆ heteroalkynyl can be a linear C₂-C₆ heteroalkynyl, a branched C₂-C₆ heteroalkynyl, a cyclic C₂-C₆ heteroalkynyl, a linear or branched C₂-C₆ heteroalkynyl, a branched or cyclic C₂-C₆ heteroalkynyl, or a linear, branched, or cyclic C₂-C₆ heteroalkynyl. Optionally, heteroalkynyl groups have two to four carbons, i.e., C₂-C₄ heteroalkynyl. In some forms, a C₂-C₄ heteroalkynyl can be a linear C₂-C₄ heteroalkynyl, a branched C₂-C₄ heteroalkynyl, a cyclic C₂-C₄ heteroalkynyl, a linear or branched C₂-C₄ heteroalkynyl, a branched or cyclic C₂-C₄ heteroalkynyl, or a linear, branched, or cyclic C₂-C₄ heteroalkynyl.

The aryl group can have six to 50 carbon atoms. A C₆-C₅₀ aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl. Optionally, aryl groups have six to 30 carbon atoms, i.e., C₆-C₃₀ aryl. In some forms, a C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Optionally, aryl groups have six to 20 carbon atoms, i.e., C₆-C₂₀ aryl. In some forms, a C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₀ aryl, or a branched fused polycyclic C₆-C₂₀ aryl. Optionally, aryl groups have six to twelve carbon atoms, i.e., C₆-C₁₂ aryl. In some forms, a C₆-C₁₂ aryl can be a branched C₆-C₁₂ aryl, a monocyclic C₆-C₁₂ aryl, a polycyclic C₆-C₁₂ aryl, a branched polycyclic C₆-C₁₂ aryl, a fused polycyclic C₆-C₁₂ aryl, or a branched fused polycyclic C₆-C₁₂ aryl. Optionally, C₆-C₁₂ aryl groups have six to eleven carbon atoms, i.e., C₆-C₁₁ aryl. In some forms, a C₆-C₁₁ aryl can be a branched C₆-C₁₁ aryl, a monocyclic C₆-C₁₁ aryl, a polycyclic C₆-C₁₁ aryl, a branched polycyclic C₆-C₁₁ aryl, a fused polycyclic C₆-C₁₁ aryl, or a branched fused polycyclic C₆-C₁₁ aryl. Optionally, C₆-C₁₂ aryl groups have six to nine carbon atoms, i.e., C₆-C₉ aryl. In some forms, a C₆-C₉ aryl can be a branched C₆-C₉ aryl, a monocyclic C₆-C₉ aryl, a polycyclic C₆-C₉ aryl, a branched polycyclic C₆-C₉ aryl, a fused polycyclic C₆-C₉ aryl, or a branched fused polycyclic C₆-C₉ aryl. Optionally, C₆-C₁₂ aryl groups have six carbon atoms, i.e., C₆ aryl. In some forms, a C₆ aryl can be a branched C₆ aryl or a monocyclic C₆ aryl.

The heteroaryl group can have three to 50 carbon atoms, i.e., C₃-C₅₀ heteroaryl. A C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. Optionally, heteroaryl groups have six to 30 carbon atoms, i.e., C₆-C₃₀ heteroaryl. In some forms, a C₆-C₃₀ heteroaryl can be a branched C₆-C₃₀ heteroaryl, a monocyclic C₆-C₃₀ heteroaryl, a polycyclic C₆-C₃₀ heteroaryl, a branched polycyclic C₆-C₃₀ heteroaryl, a fused polycyclic C₆-C₃₀ heteroaryl, or a branched fused polycyclic C₆-C₃₀ heteroaryl. Optionally, heteroaryl groups have six to 20 carbon atoms, i.e., C₆-C₂₀ heteroaryl. In some forms, a C₆-C₂₀ heteroaryl can be a branched C₆-C₂₀ heteroaryl, a monocyclic C₆-C₂₀ heteroaryl, a polycyclic C₆-C₂₀ heteroaryl, a branched polycyclic C₆-C₂₀ heteroaryl, a fused polycyclic C₆-C₂₀ heteroaryl, or a branched fused polycyclic C₆-C₂₀ heteroaryl. Optionally, heteroaryl groups have six to twelve carbon atoms, i.e., C₆-C₁₂ heteroaryl. In some forms, a C₆-C₁₂ heteroaryl can be a branched C₆-C₁₂ heteroaryl, a monocyclic C₆-C₁₂ heteroaryl, a polycyclic C₆-C₁₂ heteroaryl, a branched polycyclic C₆-C₁₂ heteroaryl, a fused polycyclic C₆-C₁₂ heteroaryl, or a branched fused polycyclic C₆-C₁₂ heteroaryl. Optionally, C₆-C₁₂ heteroaryl groups have six to eleven carbon atoms, i.e., C₆-C₁₁ heteroaryl. In some forms, a C₆-C₁₁ heteroaryl can be a branched C₆-C₁₁ heteroaryl, a monocyclic C₆-C₁₁ heteroaryl, a polycyclic C₆-C₁₁ heteroaryl, a branched polycyclic C₆-C₁₁ heteroaryl, a fused polycyclic C₆-C₁₁ heteroaryl, or a branched fused polycyclic C₆-C₁₁ heteroaryl. Optionally, C₆-C₁₂ heteroaryl groups have six to nine carbon atoms, i.e., C₆-C₉ heteroaryl. In some forms, a C₆-C₉ heteroaryl can be a branched C₆-C₉ heteroaryl, a monocyclic C₆-C₉ heteroaryl, a polycyclic C₆-C₉ heteroaryl, a branched polycyclic C₆-C₉ heteroaryl, a fused polycyclic C₆-C₉ heteroaryl, or a branched fused polycyclic C₆-C₉ heteroaryl. Optionally, C₆-C₁₂ heteroaryl groups have six carbon atoms, i.e., C₆ heteroaryl. In some forms, a C₆ heteroaryl can be a branched C₆ heteroaryl, a monocyclic C₆ heteroaryl, a polycyclic C₆ heteroaryl, a branched polycyclic C₆ heteroaryl, a fused polycyclic C₆ heteroaryl, or a branched fused polycyclic C₆ heteroaryl.

R₁ and R₂ can be independently an unsubstituted alkyl group or a substituted alkyl group.

R₁ and R₂ can be independently an unsubstituted alkyl group, such as an unsubstituted linear alkyl group. R₁ and R₂ can be independently an unsubstituted branched alkyl group. R₁ and R₂ can be independently an unsubstituted linear cyclic alkyl group. R₁ and R₂ can be independently an unsubstituted linear C₁-C₃₀ alkyl group, branched C₁-C₃₀ alkyl group, cyclic C₁-C₃₀ alkyl group, or combinations thereof. R₁ and R₂ can be independently an unsubstituted linear C₁-C₂₀ alkyl group, branched C₁-C₂₀ alkyl group, cyclic C₁-C₂₀ alkyl group, or combinations thereof. R₁ and R₂ can be independently an unsubstituted linear C₁-C₁₀ alkyl group, branched C₁-C₁₀ alkyl group, cyclic C₁-C₁₀ alkyl group, or combinations thereof. R₁ and R₂ can be independently an unsubstituted linear C₁-C₅ alkyl group, branched C₁-C₅ alkyl group, cyclic C₁-C₅ alkyl group, or combinations thereof. R₁ and R₂ can be independently an unsubstituted linear C₁-C₃ alkyl group, branched C₁-C₃ alkyl group, cyclic C₁-C₃ alkyl group, or combinations thereof. R₁ and R₂ can be independently an unsubstituted linear C₁-C₂ alkyl group, branched C₁-C₂ alkyl group, cyclic C₁-C₂ alkyl group, or combinations thereof. R₁ and R₂ can be independently an unsubstituted cyclic C₁-C₃₀ alkyl group. R₁ and R₂ can be independently an unsubstituted cyclic C₁-C₂₀ alkyl group. R₁ and R₂ can be independently an unsubstituted cyclic C₁-C₁₀ alkyl group. R₁ and R₂ can be independently an unsubstituted cyclic C₁-C₅ alkyl group. R₁ and R₂ can be independently an unsubstituted cyclic C₁-C₃ alkyl group. R₁ and R₂ can be independently an unsubstituted linear C₁-C₃₀ alkyl group. R₁ and R₂ can be independently an unsubstituted linear C₁-C₂₀ alkyl group. R₁ and R₂ can be independently an unsubstituted linear C₁-C₁₀ alkyl group. R₁ and R₂ can be independently an unsubstituted linear C₁-C₅ alkyl group. R₁ and R₂ can be independently an unsubstituted linear C₁-C₃ alkyl group. R₁ and R₂ can be independently an unsubstituted linear C₁-C₂ alkyl group. R₁ and R₂ can be unsubstituted methyl groups.

R₁ and R₂ can be independently a substituted alkyl group, such as a substituted linear alkyl group, a substituted branched alkyl group, or a substituted cyclic alkyl group. R₁ and R₂ can be independently a substituted linear C₁-C₃₀ alkyl group, branched C₁-C₃₀ alkyl group, cyclic C₁-C₃₀ alkyl group, or combinations thereof. R₁ and R₂ can be independently a substituted linear C₁-C₂₀ alkyl group, branched C₁-C₂₀ alkyl group, cyclic C₁-C₂₀ alkyl group, or combinations thereof. R₁ and R₂ can be independently a substituted linear C₁-C₁₀ alkyl group, branched C₁-C₁₀ alkyl group, cyclic C₁-C₁₀ alkyl group, or combinations thereof. R₁ and R₂ can be independently a substituted linear C₁-C₅ alkyl group, branched C₁-C₅ alkyl group, cyclic C₁-C₅ alkyl group, or combinations thereof. R₁ and R₂ can be independently a substituted linear C₁-C₃ alkyl group, branched C₁-C₃ alkyl group, cyclic C₁-C₃ alkyl group, or combinations thereof. R₁ and R₂ can be independently a substituted linear C₁-C₂ alkyl group, branched C₁-C₂ alkyl group, cyclic C₁-C₂ alkyl group, or combinations thereof. R₁ and R₂ can be independently a substituted cyclic C₁-C₃₀ alkyl group. R₁ and R₂ can be independently a substituted cyclic C₁-C₂₀ alkyl group. R₁ and R₂ can be independently a substituted cyclic C₁-C₁₀ alkyl group. R₁ and R₂ can be independently a substituted cyclic C₁-C₅ alkyl group. R₁ and R₂ can be independently a substituted cyclic C₁-C₃ alkyl group. R₁ and R₂ can be independently a substituted linear C₁-C₃₀ alkyl group. R₁ and R₂ can be independently a substituted linear C₁-C₂₀ alkyl group. R₁ and R₂ can be independently a substituted linear C₁-C₁₀ alkyl group. R₁ and R₂ can be independently a substituted linear C₁-C₅ alkyl group. R₁ and R₂ can be independently a substituted linear C₁-C₃ alkyl group. R₁ and R₂ can be independently a substituted linear C₁-C₂ alkyl group. R₁ and R₂ can be substituted methyl groups having a structure of Formula II:

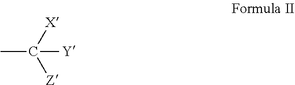

where X′, Y′, and Z′ are independently a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group;

an amino group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a hydroxyl group optionally containing one substituent at the hydroxyl oxygen, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a thiol group optionally containing one substituent at the thiol sulfur, wherein the substituent is an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a sulfonyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an amide group optionally containing one or two substituents at the amide nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof;

an azo group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an acyl group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

a carbonate ester group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an ether group containing an optionally substituted alkyl group, an optionally substituted heteroalkyl group, an optionally substituted alkenyl group, an optionally substituted heteroalkenyl group, an optionally substituted alkynyl group, an optionally substituted heteroalkynyl group, an optionally substituted aryl group, or an optionally substituted heteroaryl group;

an aminooxy group optionally containing one or two substituents at the amino nitrogen, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof; or

a hydroxyamino group optionally containing one or two substituents, wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

X′, Y′, and Z′ can be independently a hydrogen atom, a halogen atom, a nitrile group, a methyl group, or an unsubstituted aryl group. X′, Y′, and Z′ can be independently a hydrogen atom, a halogen atom, a nitrile group, or a methyl group. X′, Y′, and Z′ can be independently a hydrogen atom, a halogen atom, or a nitrile group. X′, Y′, and Z′ can be independently a hydrogen atom or a halogen atom. X′, Y′, and Z′ can be independently a hydrogen atom or a methyl group. X′, Y′, and Z′ can all be hydrogen atoms.

R₃-R₁₀ can be independently a hydrogen atom, a halogen atom, a nitrile group, a methyl group, or an unsubstituted aryl group. In some forms, when R₇ and R₈ together form an unsubstituted aryl group, R₉ and R₁₀ together do not form an unsubstituted aryl group. R₃-R₁₀ can be independently a hydrogen atom, a halogen atom, a nitrile group, or a methyl group. R₃-R₁₀ can be independently a hydrogen atom, a halogen atom, or a nitrile group. R₃-R₁₀ can be independently a hydrogen atom or a halogen atom. R₃-R₁₀ can be independently a hydrogen atom or a methyl group. R₃-R₁₀ can all be hydrogen atoms.

In a substituted group or moiety, one or more hydrogen atoms in the chemical group or moiety is replaced with one or more substituents. Any substitution is in accordance with permitted valence of the substituted atom and the substituent, and that the substitution results in a stable compound, i.e., a compound that does not spontaneously undergo transformation such as by rearrangement, cyclization, elimination, etc. Suitable substituents include, but are not limited to a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —SO₃H, —CH₂SO₂C H₃, —PO₃H₂, —OPO₃H₂, —P(═O)(OR^{T1}′)(OR^{T2}′), —OP(═O)(OR^{T1}′)(OR^{T2}′), —BR^{T1}′(OR^{T2}′), —B(OR^{T1}′)(OR^{T2}′), or -G′R^{T1}′ in which -T′ is —O—, —S—, —NR^{T2}′—, —C(═O)—, —S(═O)—, —SO₂—, —C(═O)O—, —C(═O)NR^{T2}′—, —OC(═O)—, —NR^{T2}′C(═O)—, —OC(═O)O—, —OC(═O)NR^{T2}′—, —NR^{T2}′C(═O)O—, —NR^{T2}′C(═O)NR^{T3}′—, —C(═S)—, —C(═S)S—, —SC(═S)—, —SC(═S)S—, —C(═NR^{T2}′)—, —C(═NR^{T2}′)O—, —C(═NR^{T2}′)NR^{T3}′—, —OC(═NR^{T2′})—, —NR^{T2}′C(═NR^{T3}′)—, —NR^{T2}′SO₂—, —C(═NR^{T2}′)NR^{T3}′—, —OC(═NR^{T2}′)—, —NR^{T2}′C(═NR^{T3}′)—, —NR^{T2}′SO₂—, —NR^{T2}′SO₂NR^{T3}′—, —NR^{T2}′C(═S)—, —SC(═S)NR^{T2}′—, —NR^{T2}′C(═S)S—, —NR^{T2}′C(═S)NR^{T3}′—, —SC(═NR^{T2}′)—, —C(═S)NR^{T2}′—, —OC(═S)NR^{T2}′—, —NR^{T2}′C(═S)O—, —SC(═O)NR^{T2}′—, —NR^{T2}′C(═O)S—, —C(═O)S—, —SC(═O)—, —SC(═O)S—, —C(═S)O—, —OC(═S)—, —OC(═S)O—, —SO₂NR^{T2}′—, —BR^{T2}′—, or —PR^{T2}′—; where each occurrence of R^{T1}′, R^{T2}′, and R^{T3}′ is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

Optionally, the diboronic acid compounds have a structure of Formula III:

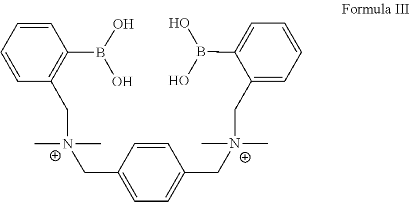 [SMILES: C[N+](C)(Cc1ccc(C[N+](C)(C)Cc2ccccc2B(O)O)cc1)Cc1ccccc1B(O)O]

Optionally, the diboronic acid compounds have a structure of Formula IV:

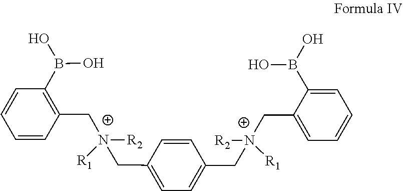

wherein R₁ and R₂ are independently an unsubstituted alkyl group, a substituted alkyl group, an unsubstituted heteroalkyl group, or a substituted heteroalkyl group, preferably an unsubstituted alkyl group or a substituted alkyl group, more preferably an unsubstituted C₁-C₁₀ alkyl group or a substituted C₁-C₁₀ alkyl group.

The diboronic acid compounds are soluble in an aqueous solution over a range of pHs, such as a pH range from about 3 to about 11.5. The aqueous solution can have a pH between about 4 and about 11.5, between about 4.5 and about 11, between about 5 and about 10.5, between about 5.5 and about 10, between about 6 and about 9.5, between about 6.5 and about 9, between about 6.5 and about 8.5, between about 6.5 and about 8, between about 6.5 and about 7.5, between about 7 and about 8, or between about 7 and about 7.5, such as 7.0, 7.1, 7.2, 7.3, 7.4, or 7.5.

The aqueous solution can be a buffer solution. Exemplary buffer solutions include, but are not limited to, phosphate buffer, phosphate buffered saline (PBS), acetate buffer, citrate buffer, maleic acid buffer, salt water, MES buffer, Bis-Tris buffer, ADA, ACES, PIPES, MOPSO, Bis-Tris propane, BES, MOPS, TES, HEPES, DIPSO, MOBS, TAPSO, Trizma, HEPPSO, POPSO, TEA, EPPS, Tricine, Gly-gly, Bicine, HEPBS, TAPS, AMPD, TABS, AMPSO, CHES, CAPSO, AMP, CAPS, CABS, or a combination thereof.

A. Counter Ions

Any of the diboronic acid compounds can also include counter ions to the tertiary amine groups (e.g. positively charged nitrogen). The counter ions can be any ions with negative charge. For example, the diboronic acid compounds of Formula I and Formula III can have counter ions to the tertiary amine groups (e.g. positively charged nitrogen).

Exemplary counter ions include, but are not limited to, halide anions (e.g. fluoride ions, chloride ions, bromide ions, or iodide ions), citrate ion, methanesulfonate ion, phosphate ion, hydrogen phosphate ion, dihydrogen phosphate ion, trihydrogen phosphate ion, bicarbonate, and combinations thereof. The counter ions can be citrate ions, methanesulfonate ions, bromide ions, or dihydrogen phosphate ions. In some forms, the counter ions are bromide ions. In some forms, the counter ions are dihydrogen phosphate ions. For example, the diboronic acid compounds of Formula III can include bromide ions on the positively charged nitrogen of the tertiary amines (referred to as DBA2+Br).

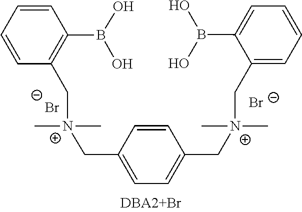 [SMILES: C[N+](C)(Cc1ccc(C[N+](C)(C)Cc2ccccc2B(O)O)cc1)Cc1ccccc1B(O)O ; [Br-] ; [Br-]]

The bromide ions of the diboronic acid compounds of Formula III can be exchanged with any counter ions with negative charge, such as the ones described above. For example, the diboronic acid compounds of Formula III can include dihydrogen phosphate ions on the positively charged nitrogen of the tertiary amines (referred to as DBA2+P).

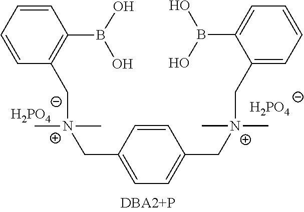

B. Binding Affinity for Glucose

The boronic acid groups (BAs) can form reversible covalent linkages to 1,2- and 1,3-diols and thus can bind sugars, such as glucose. In particular, diboronic acid compounds having structures of Formulas I and III contain two diboronic acids at a relatively restricted distance, providing high affinity for glucose binding.

The binding affinity of diboronic acid compounds for glucose can be evaluated using K_{d} values. Methods for determining K_{d} values are known in the art (see, e.g., Stootman, et al., Analyst, 131:1145-1151 (2006)). For example, the UV absorption changes of a diboronic acid compound with the increase of a sugar concentration (e.g. glucose, fructose, galactose, maltose, sucrose, and lactose) can be measured and used to calculate the K_{d} value. An exemplary calculation for K_{d} values is described in Example 3 below.

In some forms, the diboronic acid compound binds glucose with a K_{d} value between about 0.1 and about 30, between about 1 and about 10 mM, between about 2 mM and about 10 mM, or between 2 mM and about 5 mM.

For example, diboronic acid compounds of Formula I and Formula III can bind glucose with a K_{d} value between about 2 mM and about 10 mM.

C. Binding Selectivity Towards Glucose

The diboronic acid compounds show binding selectivity towards glucose compared to interference sugars, such as fructose, galactose, maltose, sucrose, lactose, or a combination thereof. For example, the diboronic acid compounds bind glucose with K_{d} value that is at least about 2-times lower compared to the K_{d} value when the diboronic acids bind to an interference sugar under the same conditions (e.g. the same temperature, pressure, solution, pH, etc).

Diboronic acid compounds having structures of Formulas I and III can bind glucose with a K_{d} value at least about 2-times lower, at least about 4-times lower, at least about 5-times lower, at least about 8-times lower, at least about 10-times lower, at least about 15-times lower, or at least about 20-times lower than a K_{d} value for the same diboronic acid binding to an interference sugar under the same conditions. For example, diboronic acid compounds of Formula III have a K_{d} value of about 1.7 mM for fructose and a K_{d} value of about 16 mM for galactose.

The diboronic acid compounds having structures of Formulas I and III can bind glucose with a K_{d} value about 1.9-times lower than the K_{d} value for fructose. The diboronic acid compounds having structures of Formulas I and III can bind glucose with a K_{d} value about 18-times lower than the K_{d} value for galactose The diboronic acid compounds having structures of Formulas I and III generally do not show affinity for maltose, sucrose, and/or lactose.

D. pKa

Depending on the pH of the aqueous solution, the hydroxyl groups of the diboronic acids of the compounds can be fully protonated, partially protonated, or fully deprotonated in an aqueous solution at a pH between about 4 and about 10.

The pKa of diboronic acid compounds can decrease upon binding with a sugar, such as glucose. For example, diboronic acid compounds can become more acidic upon diol formation (BA-diol) and result in having a lower pKa, turning neutral boronic acid groups to negatively charged BA-diol complex at a particular pH.

The diboronic acid compounds disclosed herein can show a decrease in pKa upon binding with a sugar in an aqueous solution at a pH between about 4 and about 10, between about 4.5 and about 9.5, between about 5 and about 9, between about 5 and about 8.5, between about 5 and about 8, between about 5.5 and about 8, between about 6 and about 8, between about 6.5 and about 7.5. For example, the pKa of the diboronic acids can decrease upon binding with a sugar in an aqueous solution at a pH about 7.4. For example, the pKa for the diboronic acid compounds having structures of Formulas I and III decreases upon glucose binding in an aqueous solution at a pH about 7.4. More specifically, the pKa for the diboronic acid compounds having a structure of Formula III decrease upon glucose binding in an aqueous solution at a pH about 7.4.

In some forms, the pKa value of diboronic acid can decreases by about 1 pKa units, 2 pKa units, preferably about 3 units, more preferably about 4 units upon binding with a sugar. Typically, the pKa value of the diboronic acid compounds decreases upon binding with a sugar. For example, the pKa value of the diboronic acid compounds decreases by about 1 unit, about 2 units, preferably about 3 units, optionally by about 4 units upon binding with glucose. In a particular form, the pKa value of the diboronic acid compounds decreases from about 9.4 to about 6.3 upon binding with glucose. Generally, the pKa value of diboronic acid compounds having a structure of Formula I or Formula III decreases by at least 1 unit, optionally decreases by up to about 2 units, decreases by up to about 3 units, or decreases by up to about 4 units upon binding with glucose. For example, the pKa values of diboronic acid compounds having a structure of Formula I or Formula III can decrease by at least 1 unit or at least 2 units, and up to 4 units, optionally up to about 3 units upon binding with glucose.

The diboronic acid compounds before binding with a sugar generally have a pKa value (a first pKa value) of greater than 7. Optionally, the first pKa value ranges from 7 to 11.5, from 7.5 to 11, from 8 to 10.5, from 8.5 to about 10, between about 7 and about 9.5, between about 7.5 and about 9, between about 8.5 and about 10.5, between about 9 and about 10, or between about 8.4 and about 9.4, such as 8.4, 8.5, 8.6, 8.7, 8.8, 8.9, 9.0, 9.1, 9.2, 9.3, or 9.4.

Following binding with a sugar, such as glucose, the pKa value of the diboronic acid compounds can decrease by at least 1 unit, within 1 to 2 units, or within 1 to 3 units, or within 1 to 4 units, such that the resulting pKa (a second pKa value) is between about 3 and about 10.5, between about 3.5 and about 10, between about 4 and about 10.5, between about 4.5 and about 10, between about 5 and about 9.5, between about 5 and bout 9, between about 5 and about 8.5, between about 5 and about 8, between about 5 and about 7.5, or between about 3 and about 7. For example, the diboronic acid compounds upon binding with a sugar, such as glucose, can have a resulting pKa value between about 5 and about 7 or between about 6 and about 7, such as 6.0, 6.1, 6.2, 6.3, 6.4, 6.5, 6.6, 6.7, 6.8, 6.9, or 7.0. In some forms, the diboronic acid compounds having structures of Formulas I and III after binding with a sugar, such as glucose, have a resulting pKa value of about 6.3.

For example, the pKa value of diboronic acid compounds of Formula III can decrease from being in the range of 9 to 10, such as 9.1, 9.2, 9.3, 9.4, 9.5, 9.6, 9.7, 9.8, 9.9, or 10, to being in a range from 6 to 7, such as 6.0, 6.1, 6.2, 6.3, 6.4, 6.5, 6.6, 6.7, 6.8, 6.9, or 7.0 following binding with glucose in an aqueous solution at a pH about 7.4 and room temperature.

### III. Sensors

The disclosed diboronic acid compounds can be used in any suitable sensors for the detection of glucose. Exemplary glucose sensors containing the diboronic acid compounds include conductivity sensors and optical sensors.

A. Conductivity Sensors

Conductivity sensors containing the diboronic acid compounds allow for selective detection of glucose in a sample. The conductivity sensor can be operated with low power (such as an operating voltage <20 mV) and are more energy efficient than current continuous glucose sensors. Further, the sensors can be miniaturized (e.g. to be a size of about 2 cm×2 cm or smaller) so that they can easily be worn by a subject. Optionally, the sensor is positioned at an appropriate position on the subject's body, e.g. wrist, arm, chest, abdomen, etc.

Sensors containing the disclosed diboronic acid compounds allow for the selective detection of glucose in a biological sample. Conductivity sensors containing one or more of the diboronic acid compound described herein are generally stable for at least 24 hours, optionally at least 36 hours, at least 72 hours, at least 7 days, at least 1 month, at least 2 months, or at least 3 months when in operation (operational stability). Further, the diboronic acid based conductivity sensors are generally stable for at least a year in storage (shelf-life), optionally at least 2 years in storage or at least 3 years in storage at room temperature, or at least 3 years, at least 4 years, or at least 5 years under cold storage (such as between 2-8° C.). “Stability” and “stable” refers to a sensor's capability to preserve at least about 80% of its original signal in response to a target at the same concentration and under the same conditions (e.g. the same temperature, pressure, solution, pH, etc).

The conductivity sensor generally includes a reservoir containing the diboronic acid compound(s) and a buffer solution or buffer salts, a pair of electrodes, a membrane, and optionally a detector. The electrodes are in electrical communication with each other.

When a buffer solution is in the reservoir, the diboronic acid compound(s) are in the buffer solution and an electrically conductive surface of each electrode is in contact with the buffer solution. The membrane is configured to prevent or reduce ion exchange between the buffer solution and the biological sample.

When buffer salts are in the reservoir, the diboronic acid compound(s) and buffer salt(s) are in a solid form, optionally in the form of a powder, film, or tablet. In these conductivity sensors, a solvent, such as water or an aqueous solvent, is added to dissolve the diboronic acid compound(s) and buffer salt(s) to form a buffer solution prior to using the sensor. Then the electrically conductive surface on each electrode is in contact with the formed buffer solution.

The membrane is typically is bipolar membrane described below.

The biological sample contains glucose of unknown concentration. The biological sample is added into the buffer solution and thereby forms a test sample.

The sample reservoir is typically defined by side walls and a bottom surface, and contains an opening configured to allow the biological sample to enter the reservoir. At least a portion of the bottom surface and/or one or both of the side walls of the reservoir is formed from the electrically conductive surface of each of the electrodes. Optionally, the electrically conductive surfaces of the electrodes are located on and form part of the bottom surface of the reservoir.

The membrane is located adjacent to the opening of the reservoir, and defines an outer surface that encloses the buffer solution or solid buffer salts and compound inside of the reservoir. The membrane forms a top portion that is able to selectively filter out interfering materials (such as cations and anions and/or macrosolutes (i.e. solutes of molecular weight of the order of 500 Da or higher)) present in the biological sample (e.g. blood) so that they do not enter the reservoir.

Generally, any diboronic acid compounds of Formulae I-IV can be used in the conductivity sensor. The diboronic acid compounds included in the conductivity sensors can have the same structures or different structures. In some embodiments, the conductivity sensor contains the diboronic acid compounds of Formula III only. In some forms, the sensor includes two or more different diboronic acid compounds. Optionally, more than one sensor is provided in a set, such as in an array (e.g. a conductivity sensing array). In some embodiments, each sensor in the set of sensors contains the same diboronic acid compound(s). In some embodiments, at least one sensor in the set of sensors contains a different diboronic acid compound from the compound in another sensor in the set, i.e. at least one of the diboronic acid compounds has a different structure compared to the diboronic acid compound(s) in the other sensors in the set.

Exemplary biological samples include bodily fluids such as such as interstitial fluid, saliva, sputum, tear, sweat, urine, exudate, whole blood, serum, plasma, mucus or vaginal secretion. Optionally, the biological samples are processed and then added into a buffer solution to form the test sample.

Optionally, the conductivity sensor contains a detector in electrical communication with the electrode(s). The detector measures the electrochemical signal. Detectors for measuring electrochemical signals are known. For example, the electrochemical signal can be measured by a miniaturized potentiostat.

Optionally, the conductivity sensor contains a sample reservoir to retain the buffer solution or test sample. The sample reservoir can be made from any suitable inert material, such as plastic, glass, or a polymeric material, such as polydimethylsiloxane (PDMS).

In some embodiments, two or more conductivity sensors can be combined to form a conductivity sensing array. Each sensor in the conductivity sensing array can contain the same or different diboronic acid compounds. In some embodiments, each conductivity sensor in the sensing array contains the same diboronic acid compounds. In some embodiments, at least one of the conductivity sensors in the sensing array contains a different diboronic acid compound from the other sensors, optionally the array includes three or more sensors containing different diboronic acid compounds. For example, two or more conductivity sensors in the sensing array contain a first diboronic acid compound and at least one conductivity sensor in the sensing array contains a second diboronic acid compound that is different from the first diboronic acid compound.

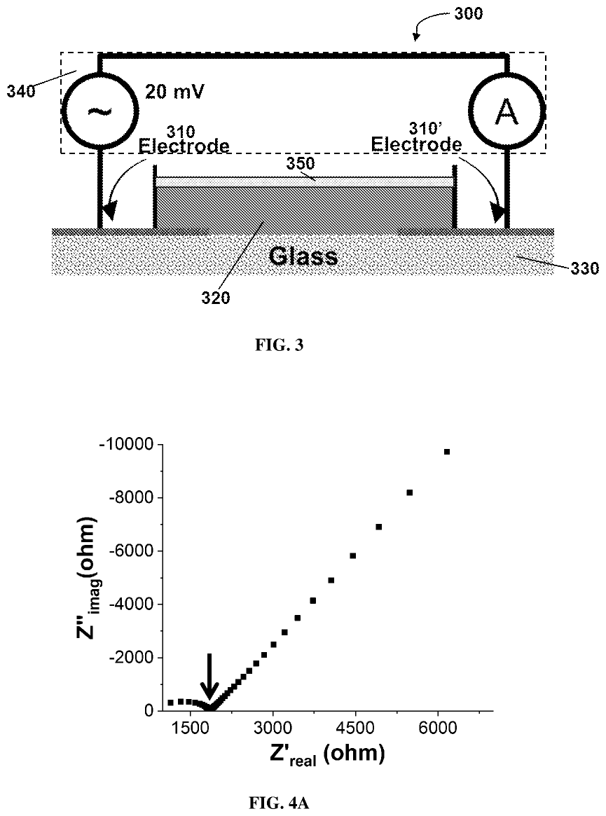

An exemplary conductivity sensor 300 is depicted in FIG. 3. The conductivity contains a pair of electrodes 310 and 310′ in electrical communication with a detector 340, e.g. electrically connected to the detector, and the detector 340 can measure the conductivity change. In FIG. 3, the detector is depicted by dashed lines and includes at least a resistor, an amp meter, connected by conductive material (e.g. wires). The electrodes 310 and 310′ are supported on a glass substrate 330 and placed apart to prevent a short circuit. Diboronic acid compounds (not shown) are located within a sample reservoir 320. The sample reservoir 320 retains a buffer solution and is arranged such that an electrically conductive surface on each of electrode 310 and electrode 310′ is in contact with the buffer solution. The diboronic acid compounds are soluble in the buffer solution. A membrane 350 is placed adjacent to the opening of the reservoir 320, and defines an outer surface that encloses the buffer solution or solid buffer salts and compound inside of the reservoir.

1. Electrode

The electrodes of the conductivity sensors can be any substance that is capable of conducting an electric current. Optionally, two electrodes are included in the conductivity sensor that are in electrical communication with each other and typically placed apart to avoid short circuits. The two electrodes can be kept at a distance (i.e., the inter-electrode gap) between about 1 μm and about 10 cm. The inter-electrode gap can be varied to mitigate ohmic resistance losses.

Typically, the sensor surfaces do not absorb organic molecules. In some forms, the conductivity sensors further contain a substrate, where the electrodes are supported on a substrate having a planar surface such as a pad or a patch. The substrate supporting the electrodes in the conductivity sensor can be non-conductive or have a portion that is conductive. In some forms, the electrodes can be deposited on the substrate by coating, such as by spin-coating, drop-casting, or electropolymerization (i.e. electropolymerization on pre-patterned substrate that has a conductive portion).

The electrodes included in the conductive sensors can be made from the same materials or different materials. In some forms, the two electrodes included in the conductivity sensor are made from the same material, e.g. platinum. In some forms, the two electrodes included in the conductivity sensor are made from different materials, e.g. one electrode is made from a first material, such as platinum, and the other electrode is made from a second material, such as gold.

The electrodes included in the conductivity sensors can be organic or inorganic in nature, as long as they are able to conduct electrons through the material. The electrodes included in the conductivity sensors can be a polymeric conductor, a metallic conductor, a semiconductor, a carbon-based material, a metal oxide, or a modified conductor. The electrodes can be in any suitable form such as a film, a mesh, a rod, or a disk. The electrodes can have any suitable cross-sectional shape such as regular shapes including, but not limited to, square, circle, oval, triangle, and rectangle, and irregular shapes such as a waveform. In some forms, the electrodes are printed electrodes made of metals. In some forms, the printed electrodes are suitable for a single use. In others, it is suitable for cleaning and can be used more than one time.

The electrodes in the conductivity sensors can be made of a metallic conductor. Suitable metallic conductors include, but are not limited to, gold, chromium, platinum, iron, nickel, copper, silver, stainless steel, mercury, tungsten and other metals suitable for electrode construction. The metallic conductor can be a metal alloy, optionally made of a combination of metals disclosed herein. Conductive substrates, which are metallic conductors, can be constructed of nanomaterials made of gold, cobalt, diamond, and other suitable metals. Optionally, the conductive substrate is platinum, gold or silver.

The electrodes in the conductivity sensors can be made from carbon-based materials. Exemplary carbon-based materials are carbon cloth, carbon paper, carbon screen printed electrodes, carbon paper, carbon black, carbon powder, carbon fiber, singe-walled carbon nanotubes, double-walled carbon nanotubes, multi-walled carbon nanotubes, carbon nanotube arrays, diamond-coated conductors, glassy carbon and mesoporous carbon. In addition, other exemplary carbon-based materials are graphene, graphite, uncompressed graphite worms, delaminated purified flake graphite, high performance graphite and carbon powders, highly ordered pyrolytic graphite, pyrolytic graphite, and polycrystalline graphite. In some forms, the conductive substrate can be printed carbon. In some forms, the conductive substrate can be glassy carbon.

The electrodes in the conductivity sensors can be a semiconductor. Suitable semiconductors are prepared from silicon and germanium, which can be doped (i.e., the intentional introduction of impurities into an intrinsic semiconductor for the purpose of modulating its electrical and structural properties) with other elements. The semiconductors can be doped with phosphorus, boron, gallium, arsenic, indium, antimony, or combinations thereof.

The electrodes in the conductivity sensors can be a metal oxide, metal sulfide, main group compound, or modified materials. Exemplary conductive substrates of this type include, but are not limited to, indium-tin-oxide (ITO) glass, nanoporous titanium oxide, tin oxide coated glass, cerium oxide particles, molybdenum sulfide, boron nitride nanotubes, aerogels modified with a conductive material such as gold, solgels modified with conductive material such as carbon, ruthenium carbon aerogels, and mesoporous silicas modified with a conductive material such as gold. In some forms, the conductive substrate is ITO glass.

In some forms, the electrodes included in the conductivity sensors contain one or more conducting materials. In forms where the conductive substrate contains two or more conducting materials, the first conducting material can be a conducting polymer and the second conducting material can be a different type of conducting material. Suitable conducting polymers include, but are not limited to, poly(fluorine)s, polyphenylenes, polypyrenes, polyazulenes, polynaphthalenes, poly(pyrrole)s, polycarbozoles, polyindoles, polyzaepines, polyanilines, poly(thiophene)s, poly(3,4-ethylenedioxythiophene), poly(p-phenylene sulfide), poly(acetylene)s, poly(p-phenylene vinylene), and polyimides. The second conducting material can be sputter-coated on top of the first conducting polymer, such that the aggregate of the two conducting materials form the conductive substrate.

2. Buffer Solution

Optionally, the conductivity sensor includes a buffer solution. The buffer solution contains ions, atoms, or molecules that have lost or gained electrons, and is electrically conductive. The buffer solution in the conductivity sensors is in contact with a conductive surface of each electrode. The buffer solution contains a diboronic acid compound, where the diboronic acid compound is soluble in the buffer solution. Typically, the diboronic acid compound has a solubility of at least about 1 g/L in the buffer solution at pH about 7.4 and 25° C.

Optionally, the conductivity sensor includes the diboronic acid compound(s) and buffer salt(s) in solid form, such as in the form of a powder, film, or compressed tablet, optionally in powdered form, in the sample reservoir. In these cases, a solvent, such as water or an aqueous solution, is added to dissolve the diboronic acid compound(s) and buffer salt(s) to form a buffer solution prior to using the sensor. The ratio in mole between the buffer salt(s) and the diboronic acid compound(s) may be between 20 and 5, between 15 and 5, such as 10.

The buffer solution can be an aqueous solution. Exemplary buffer solutions included in the conductivity sensors include, but are not limited to, phosphate buffer, phosphate buffered saline (PBS), acetate buffer, citrate buffer, maleic acid buffer, salt water, MES buffer, Bis-Tris buffer, ADA, ACES, PIPES, MOPSO, Bis-Tris propane, BES, MOPS, TES, HEPES, DIPSO, MOBS, TAPSO, Trizma, HEPPSO, POPSO, TEA, EPPS, Tricine, Gly-gly, Bicine, HEPBS, TAPS, AMPD, TABS, AMPSO, CHES, CAPSO, AMP, CAPS, CABS, or combinations thereof.

Generally, the diboronic acid compound is present in the buffer solution in a concentration of between about 0.1 mM and about 100 mM, between about 0.5 mM and about 50 mM, between about 1 mM and about 10 mM, or between about 1 mM and about 5 mM, such as a concentration of about 4.5 mM.

The accuracy of the conductivity sensors can be affected by carbon dioxide (CO₂). CO₂ in solution can form carbonic acid, which decreases the pH and increases the background conductance (Arnold, et al., J. Membr. Sci., 167:227-239 (2000)). Generally, the buffer solution can maintain a desired pH or pH range and does not generate a large background conductance. The buffer solution typically has a pH of between about 3 and about 11.5, between about 4 and about 11.5, between about 4.5 and about 11, between about 5 and about 10.5, between about 5.5 and about 10, between about 6 and about 9.5, between about 6.5 and about 9, between about 6.5 and about 8.5, between about 6.5 and about 8, between about 6.5 and about 7.5, between about 7 and about 8, or between about 7 and about 7.5. Preferably, the buffer solution has a pH of about 7.4.

In some forms, the buffer solution is phosphate buffer solution containing H₂PO₄⁻/HPO₄²⁻ ions. In some forms, the buffer solution is phosphate buffer solution containing about 2 mM H₂PO₄⁻ and about 2.5 mM HPO₄²⁻. In some forms, the buffer solution is bicarbonate buffer solution containing about 1.6 mM H₂CO₃ and about 16 mM HCO₃⁻.

3. Processors

Optionally, the conductivity sensor includes a processor. The processor performs mathematical analysis using an appropriate algorithm or signal processing on the electrical data measured by the detector and calculates the glucose concentration in the test sample. Suitable processors that can be included in the conductivity sensor include commercially available processors.

In some embodiments, the processor is a microprocessor board which can be integrated in the conductivity sensor. For example, the processor can be integrated in the detector, which is in electrical communication with the electrodes in the senor.

Optionally, the processor in the conductivity sensor can transmit one or more signals or data to an output device by a wireless transmitter. In some embodiments, the processer can store data. Optionally, the processor is detached from the conductivity sensor and transfers data to an output device, such as a computer.

4. Output Devices

Optionally, the conductivity sensor includes an output device. The output(s) from the processor (i.e. calculation results) can be transmitted to an output device and visually displayed on a user interface of the output device, and/or converted to a sound, and/or a vibration of the output device. Suitable output devices that can be included in the conductivity sensor include a computer, watch, smart phone, personal digital assistant, exercise equipment, etc.

In some embodiments, the output device is portable and powered by a power source. Optionally, the power source is a single use or rechargeable battery.

The output device and the processor are typically in electrical communication, such as wireless electrical communication. For example, the output device can include a short-range wireless transceiver which is a transmitter operating on a wireless protocol, e.g. Bluetooth, part-15, or 802.11. “Part-15” refers to a conventional low-power, short-range wireless protocol, such as that used in cordless telephones. The short-range wireless transmitter, e.g., a BLUETOOTH transmitter, receives information from the processor and transmits this information in the form of a packet through an antenna. An external laptop computer or hand-held device features a similar antenna coupled to a matched wireless, short-range receiver that receives the packet. An exemplary hand held device is a cellular telephone with a Bluetooth circuit integrated directly into a chipset used in the cellular telephone. In this case, the cellular telephone may include a Software application that receives, processes, and displays the information.

Optionally, the wireless component is a long-range wireless transmitter that transmits information over a terrestrial, satellite, or 802.11-based wireless network. Suitable networks for long-range wireless transmitters include those operating one or more of the following protocols: CDMA, GSM, GPRS, Mobitex, DataTac. iDEN, and analogs and derivatives thereof. Alternatively, the handheld device is a pager or PDA.

B. Optical Sensor

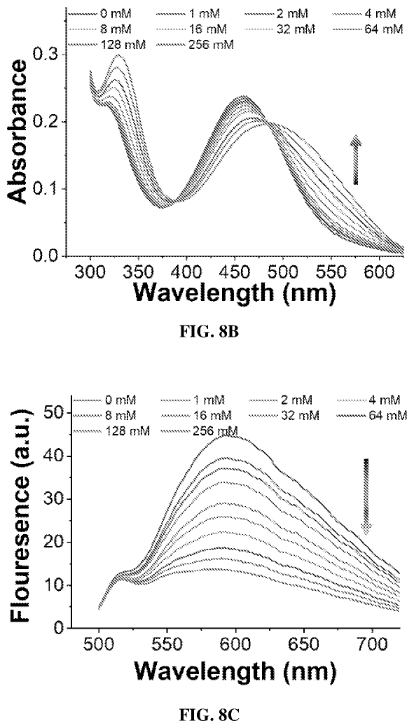

Optical sensors containing the disclosed diboronic acid compounds are also provided. The optical sensor typically contains (1) a diboronic acid compound, (2) a dye, (3) a light source, and (4) a detector. The diboronic acid compound and the dye form a complex (DBA-D complex). In the presence of glucose, the dye in the DBA-D complex can be replaced by glucose, which results in a change in the optical signals of the DBA-D complex, such as absorbance, fluorescence, or both absorbance and fluorescence. (see FIGS. 8B and 8C).

Optionally, the optical sensor includes a buffer solution, and the DBA-D complex is soluble in the buffer solution. The buffer solution can be any buffer solution described herein.

Optionally, the optical sensors also include a processor, a transmitter, and/or an output device as described above.

1. DBA-D Complexes

The optical sensor includes a DBA-D complex formed form a diboronic acid compound and a dye. The concentration ratio of the dye to the diboronic acid compound forming the DBA-D complex can be in a range from 1:0.1 to 1:10, from 1:0.5 to 1:10, from 1:1 to 1:10, from 1:0.1 to 1:5, or from 1:0.1 to 1:1, such as 1:0.5.

Any diboronic acid compounds of Formulae I-IV can be used in the optical sensor to form a complex with a dye. Optionally, two or more diboronic acid compounds having different structures are included in the optical sensor. In some embodiments, the diboronic acid compounds included in the optical sensors have the same chemical structures. In some embodiments, the diboronic acid compounds included in the optical sensors are different, e.g. the optical sensors include a first diboronic acid compound and at least a second diboronic acid compound is different than the first compound. In a particular form, the optical sensor contains diboronic acid compounds of Formula III.

The dye included in the optical sensors that forms a complex with the diboronic acid compound can be any molecule that emits fluorescence and/or absorbs light at a wavelength upon binding with the diboronic acid compound(s) in the sensor. Exemplary dyes that can be included in the optical sensors include, but are not limited to, Alizarin Red S (ARS), pyrocatechol violet, and esculetin. For example, ARS can form a complex with the diboronic acid of Formula III (ARS-DBA2+) and emit fluorescence with a peak around 600 nm (see FIG. 8C). Glucose can replace the ARS in the ARS-DBA2+ complex, resulting in a decrease of the fluorescence signal. Alternatively or additionally, the dye included in the optical sensors can form a DBA-D complex with the diboronic acid compound, which shows a change in absorbance signal, indicating replacement of the dye with glucose in the DBA-D complex (see FIG. 8B).

2. Light Sources and Detectors

Suitable light sources that can be in the optical sensors include a light emitting diode (LED) or another light source that emits radiation, including radiation over a range of wavelengths that activates the DBA-D complex. For example, the light source in the optical sensors can emit radiation at a wavelength that causes the DBA-D complex to fluoresce. Alternatively or additionally, the light source in the optical sensor emits radiation over a range of wavelengths, which causes the DBA-D complex to absorb the radiation at a specific wavelength within the range of radiation wavelengths.

The detector(s) included in the optical sensor is sensitive to light emitted and/or absorbed by the DBA-D complex such that a signal is generated by the detector in response thereto. A change in the signal upon glucose binding with the diboronic acid compound to replace the dye in the DBA-D complex is indicative of the presence and/or the level of glucose. Suitable detectors that can be included in the optical sensors include, but are not limited to, photodiodes, phototransistors, photoresistors, or other photosensitive elements.

3. Exemplary Optical Sensors

Exemplary optical sensors using the disclosed diboronic acid compounds can have the same or a similar structure to the Eversense® fluorescence sensors (i.e. having the same arrangement for light source and detectors, and optionally processors and output devices, but use the DBA-D complex in place of the indicator molecules). Exemplary set ups for the physical components of these optical sensors are described in U.S. Pat. No. 9,743,869 to Caban; U.S. Pat. No. 9,693,714 to DeHennis and Colvin; U.S. Pat. No. 9,498,156 to Whitehurst and Huffstetler; U.S. Pat. No. 7,822,450 to Colvin, et al.; U.S. Pat. No. 7,227,156 to Colvin, et al.; U.S. Pat. No. 7,157,723 to Colvin, et al.; and U.S. Pat. No. 7,800,078 to Colvin, et al.

An exemplary optical sensor 1000 is depicted in FIG. 11. Optical sensor 1000 includes a sensor housing 1020 (i.e., body, shell, sleeve, or capsule). The sensor housing 1020 may be formed from a suitable, optically transmissive polymer material, for example, acrylic polymers (e.g., polymethylmethacrylate (PMMA)). The sensor 1000 includes DBA-D complex 1040. Sensor 1000 may include a matrix layer 1060 (i.e., graft or gel) coated on or embedded in at least a portion of the exterior surface of the sensor housing 1020, with the DBA-D complex 1040 distributed throughout the matrix layer 1060. The matrix layer 1060 may cover the entire surface of sensor housing 1020 or one or more portions of the surface of housing 1020. DBA-D complex 1040 may be distributed throughout the entire matrix layer 1060 or only throughout one or more portions of the matrix layer 1060. Alternatively, the matrix layer 1060 may be disposed on the outer surface of the sensor housing 1020 in other ways, such as by deposition or adhesion. Optionally, the optical sensor does not include a matrix layer 1060 and the DBA-D complex 1040 are coated on the surface of the sensor housing 1020.

The sensor 1000 includes a light source 1080 that emits radiation over a range of wavelengths that interact with the DBA-D complex 1040. For example, in the case of a fluorescence-based sensor, light source 1080 emits radiation at a wavelength which causes the DBA-D complex 1040 to fluoresce. Sensor 1000 also includes one or more photodetectors, collectively 1110 which, in the case of a fluorescence-based sensor, is sensitive to fluorescent light emitted by the DBA-D complex 1040 such that a signal is generated by the photodetector 1110 in response thereto that is indicative of the level of fluorescence of the DBA-D complex. Sensor 1000 may also include one or more optical filters, collectively 1120, such as high pass or band pass filters. The one or more optical filters 1120 may cover a photosensitive side of the one or more photodetectors 1110. The optical filter 1120 may cover all of the one or more photodetectors 1110. Alternatively, each of the optical filters 1120 may correspond to only one of the photodetectors 1110 and cover only the one of the photodetectors 1110. The optical filters 1120 may prevent or substantially reduce the amount of radiation generated by the light source 1080 from impinging on a photosensitive side of the photodetectors 1110. At the same time, the optical filters 1120 may allow light (e.g., fluorescent light) emitted by the DBA-D complex 1040 to pass through and strike the photosensitive side of the photodetectors 1110. This reduces “noise” attributable to incident radiation from the light source 1080 in the light measurement signals output by the photodetectors 1110.

The sensor 1000 optionally includes an inductive element 1140 (i.e. a processor and/or transmitter) to communicate information to an external output device (not shown).

Sensor 1000 may include a semiconductor substrate 1160 that contains circuitry to provide communication paths between the various components. The photodetectors 1110 may be mounted on the semiconductor substrate 1160 or fabricated in the semiconductor substrate 1160. The light source 1080 may be mounted on the semiconductor substrate 1160 or fabricated in the semiconductor substrate 1160. Sensor 1000 may include one or more capacitors (1180a, 1180b, 1180c) collectively 1180. The one or more capacitors 1180 may be, for example, antenna tuning capacitors and/or one or more regulation capacitors. Sensor 1000 may also include a reflector (i.e., mirror) 1190 attached to the semiconductor substrate 1160 at an end thereof, such that a face portion 1210 of reflector 1190 is generally perpendicular to a top side of the semiconductor substrate 1160. The face 1210 of the reflector 1190 may reflect radiation emitted by light source 1080 and block radiation emitted by light source 1080 from entering the axial end of the sensor 1000. Alternatively, the reflector 1190 may be mounted on the top side of the semiconductor substrate 1160 (e.g., in a groove on the top side thereof) and serve the same function.

4. Optical Sensing Arrays

In some embodiments, two or more optical sensors can be used together as an optical sensing array. The optical sensing array can contain the same or different DBA-D complexes. A different DBA-D complex means that the structure or concentration of the diboronic acid compound and/or the dye forming a first DBA-D complex is different from that forming a second DBA-D complex. In some embodiments, each of the optical sensors in the sensing array contains the same DBA-D complex. In some forms, the optical sensors in the sensing array contains different DBA-D complexes. For example, two or more optical sensors in the sensing array contain a first DBA-D complex and at least one optical sensor in the sensing array contains a second DBA-D complex that is different from the first DBA-D complex.

C. Continuous Glucose Monitoring System

Continuous glucose monitoring systems (CGMS) can include one or more sensors described above, which contain one or more of the diboronic acids described herein. The CGMS can be used as a continuous sensing system that measures the concentration of glucose in a body fluid (e.g. blood, serum, plasma, interstitial fluid, cerebral spinal fluid, lymph fluid, ocular fluid, saliva, or oral fluid) of a mammal, such as a human.

The CGMS can be configured to be applied on a permeabilized skin site of the mammal (e.g. the human), such as one which has been abraded or permeabilized by sonophoresis or iontophoresis. Alternatively, the CGMS may include a component for extracting interstitial fluid and/or blood from the patient, such as a plurality of microneedles. Optionally, the CGMS is implanted under the skin of a mammal (e.g. a human), such that the interstitial fluid and/or blood flows into the sensor of the CGMS.

Optionally, the CGMS contains a membrane, such as a bipolar membrane, that blocks the interferences, such as cations and anions and/or macrosolutes (i.e. solutes of molecular weight of the order of 500 Da or higher) present in the body fluid (e.g. blood) from entering the sensor.

In use, glucose in the body fluid transfers from the patient's body into the CGMS, binds with the diboronic acid compound, and produces a change in electrical and/or optical signal.

Optionally, a processor in the sensor of the CGMS can process the electrical and/or optical signal associated with glucose binding, calculate the glucose concentration in the mammal (e.g. in the blood of the mammal), and transmit the calculated results to an output device, producing a visual display, a sound, and/or a vibration of the output device.

Optionally, the CGMS includes one or more conductivity sensors or one or more optical sensors described above, and optionally a bipolar membrane, and/or a plurality of microneedles for fluid extraction. In some embodiments, the CGMS contains one or more conductivity sensors described above, and optionally a bipolar membrane and/or a plurality of microneedles for fluid extraction. In some embodiments, the CGMS contains one or more optical sensors described above, and optionally a bipolar membrane and/or a plurality of microneedles for fluid extraction.

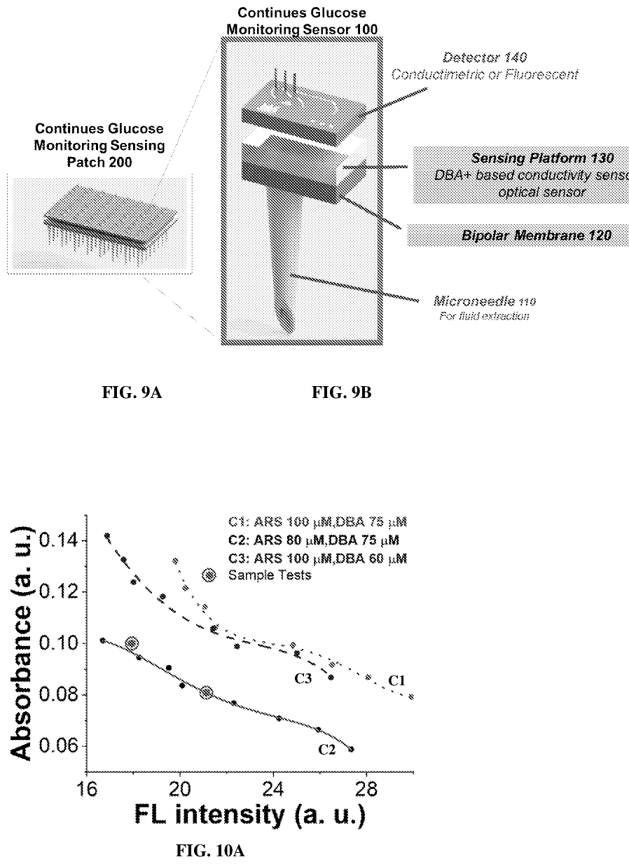

An exemplary CGMS in the form of a patch 200 is depicted in FIG. 9A. FIG. 9B provides an exploded view of a single hollow microneedle and the corresponding sensor, which is part of the patch depicted in FIG. 9A. The CGMS patch contains a plurality of hollow microneedles and an array of CGM sensors 100. The microneedles are typically 50-900 microns in length and can be formed from any suitable inert material, such as silicon, titanium, stainless steel, or inert polymers. The CGM sensor includes a microneedle 110, a bipolar membrane 120, and a sensing platform 130 containing one or more conductivity sensors and/or one or more optical sensors. The CGM sensor 100 further contains a detector 140 to measure the conductivity and/or fluorescence in situ. Methods and detectors for measuring electrochemical signal and optical signal are known. For example, the electrochemical signal can be measured by a miniaturized potentiostat. The CGMS patch 200 can be placed on and attach to a surface on the skin of a subject. The subject can be a human or other mammal.

The bipolar membrane can be used to block the interferences present in biologic milieu/media, such as blood. The bipolar membrane is typically an ion exchange membrane possessing transport properties that can be freely and selectively permeable to common water-soluble blood plasma microsolutes, such as glucose and other mono- and di-saccharides, urea, and the like. In some forms, the bipolar membrane can be a bilayer laminate containing a thin film of a strong-base, high-ion-density anion exchanger, and a thin film of a strong-acid, high-ion-density cation exchanger, strongly bonded to one another with a high-water-content adhesive (Simons, et al., J. Membr. Sci., 78:13 (1993)). Films of anion exchanger and cation exchanger are known, such as NeoSepta® produced by Tokuyama Soda (Japan) (see, e.g., U.S. Pat. No. 7,499,738 to Gerber, et al.). For example, Donnan co-ion exclusion prevents entry of anions into the cation exchange layer and of cations into the anion exchange layer. If the ionic strength of the contacting solution is much lower than that of the ion exchangers of the membrane, there will be no passage of either cations or anions across the membrane. In addition, since the membrane is highly hydrated (i.e. water content is in the range of about 50% by volume or more), any nonionic microsolute can freely pass through both layers of the laminate. Since the ion exchange layers of the laminate are typically highly cross-linked to prevent osmotic swelling in aqueous media, the membrane can also be expected to be impermeable to nonionic macrosolutes (i.e. solutes of molecular weight of the order of 500 Da or higher).

The microneedles are typically hollow microneedles. The microneedles can be formed of any suitable material, such as can be embedded in the skin of a subject and attach the GCMS patch on the subject's body part. Body fluid, such as interstitial fluid, is extracted through the microneedles and transported to pass through the bipolar membrane, reaching the sensing platform containing the conductivity sensor or optical sensor.

### IV. Methods of Making the Diboronic Acid Compound

Disclosed are methods of making the disclosed diboronic acid compounds. In some forms, methods of making the compounds of Formula I and III can involve:

(a) performing a reaction between a compound of Formula V and a compound of Formula VI; and

(b) performing a reaction between the adduct from step (a) and a compound of Formula VII.

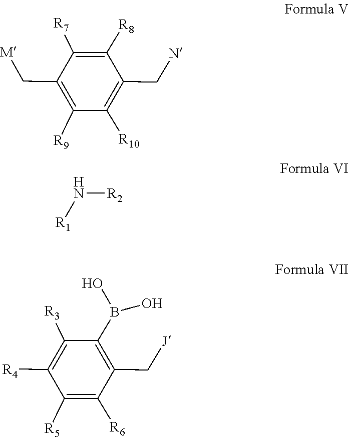

where R₁-R₁₀ are as defined above; and

where M′, N′, and J′ are independently a halogen atom (such as fluorine, chlorine, bromine, or iodine), hydroxyl group, sulfydryl group, aldehyde group, or carboxyl group.

Steps (a) and (b) are performed in an organic solvent. The organic solvent in step (a) and step (b) can be the same or different. Exemplary organic solvents include, but are not limited to, dimethyl sulfoxide, methylene chloride, chloroform, tetrahydrofuran (THF), acetone, dioxane, ethyl acetate, dimethylene carbonate, dimethyl formamide (DMF), methyl ethyl ketone, butyl acetate, butyl propionate, and diethyl carbonate. Optionally, the adduct of step (a) is dried and dissolved in an organic solvent that is different from the organic solvent in step (a) to perform the reaction of step (b). For example, the adduct of step (a) in THF is dried and dissolved in DMF for the reaction of step (b). The adduct of step (a) can be dried by removing solvent under rotary evaporation.

The reaction of step (a) can be performed at a first reaction temperature over a suitable time period to form the adduct. For example, when M′ and N′ are independently a halogen atom, the reaction of step (a) is performed at a temperature between about −78° C. and about 100° C., between about −70° C. and about 95° C., between about −65° C. and about 90° C., between about −60° C. and about 85° C., between about −55° C. and about 80° C., between about −50° C. and about 75° C., between about −45° C. and about 70° C., between about −40° C. and about 65° C., between about −35° C. and about 60° C., between about −30° C. and about 55° C., between about −25° C. and about 50° C., between about −20° C. and about 45° C., between about −15° C. and about 40° C., between about −10° C. and about 35° C., between about −5° C. and about 30° C., between about 0° C. and about 25° C., between about 5° C. and about 20° C., or between about −78° C. and about 25° C. for a time period between about 10 minutes and about 5 hours, between about 10 minutes and about 4 hours, between about 10 minutes and about 3 hours, between about 10 minutes and about 2 hours, or between about 10 minutes and about 1 hour. Optionally, the compound of Formula V is added in an organic solvent containing the compound of Formula VI at a temperature below about −50° C., below about −60° C., or below about −78° C., where the temperature is warmed to a reaction temperature described above.

The reaction of step (b) can be performed at a second reaction temperature over a suitable time period. For example, when J′ is a halogen atom, the reaction of step (b) can be performed at a reaction temperature between about 20 and about 100° C., between about 25° C. and about 95° C., between about 30° C. and about 90° C., between about 35° C. and about 85° C., between about 40° C. and about 80° C., between about 45° C. and about 75° C., between about 40° C. and about 70° C., 35° C. and about 65° C., 40° C. and about 60° C., or between about 45° C. and about 55° C. in a time period between about 10 hours and about 24 hours, between about 12 hours and about 22 hours, between about 14 hours and about 20 hours, between about 16 and about 24 hours, or between about 15 hours and about 18 hours.

Optionally, the reaction product of step (b) is washed with a washing solvent to remove impurities. The washing step can occur one or more times, such as once, twice, three times, four times, or five times. Exemplary washing solvents include, but are not limited to, ethyl acetate, ether, acetone, acetonitrile, THF, dioxane, dimethyl ether, dichloromethane, and chloroform. Optionally, the washed reaction product is then dried, such as air-dried, via rotary evaporation, freeze-dried, or dried in a vacuum oven at a temperature between about 40° C. and about 80° C.

Generally, in bench scale processes, the compound of Formula V and the compound of Formula VI are present in a mole ratio that is equal to or lower than 1:3. The amount of the adduct from step (a) and compound of Formula VII generally is present in a mole ratio that is equal to or lower than 1:2.5.

An exemplary method for making the diboronic acid compound of Formula III is described in Example 1. Briefly, 1,4-dibromomethyl benzene reacts with dimethyl amine in tetrahydrofuran; a subsequent reaction with 2-bromomethylphenyl boronic acid results in the diboronic acid compound of Formula III with bromide counter anions (DBA2+Br).

The counter ions on the diboronic acid compounds can be exchanged with another counter ion. For example, the bromide ions in the diboronic acid compound (DBA2+Br) can be replaced with dihydrogen phosphate ions (DBA2+P) through anion exchange, such as simple reverse column in H₃PO₄ solution. For example, the counter ions can be exchanged by simply mixing DBA2+Br with another salt in solution.

### V. Methods of Using the Diboronic Acid Compound

The disclosed diboronic acid compounds can be used to detect the presence, the absence, and/or the concentration of glucose in a sample using a conductivity sensor, optical sensor, or CGMS.

Sensors containing the diboronic acid compounds, such as the conductivity sensor and optical sensor described above, can be used for both in vitro and in vivo applications. In some forms, the conductivity sensors and/or optical sensors can be miniaturized and portable. In some forms, the sensor is small enough to be applied onto a medical device. In some forms, the sensor is wearable or attachable to a subject, such as a CGMS patch.

The conductivity sensor and optical sensor can be connected to an acquisition system, such as a potentiostat, and, optionally, to a display system. The display system may be a portable display system with a screen to display the sensor readings or calculated results. Portable display systems include smartphones, tablets, laptops, desktop, pagers, watches, and glasses.

The sensors permit non-invasive testing of the presence, absence, and/or concentration of glucose in a test sample. Exemplary biological samples include bodily fluids such as such as interstitial fluid, saliva, sputum, tear, sweat, urine, exudate, whole blood, serum, plasma, mucus or vaginal secretion. In some forms, the biological samples are processed or unprocessed and added into a buffer solution to form the test sample. In some forms, the sensors permit semi-invasive testing of the presence, absence, or concentration of glucose in a test sample. Typically, the sensors can detect glucose from 0 to about 30 mM, from about 5 mM to about 20 mM, from about 12 mM to about 30 mM, or from about 2 mM to about 30 mM.

Typically, the volume of test sample for measurement can be between about 0.1 μL and about 1 mL. In some instances, the volume of test sample is between about 0.1 μL and about 100 μL, between about 0.1 μL and about 50 μL, between about 0.1 μL and about 30 μL, between about 1 μL and about 30 μL, between about 10 μL and about 30 μL.

A. Conductivity Sensor

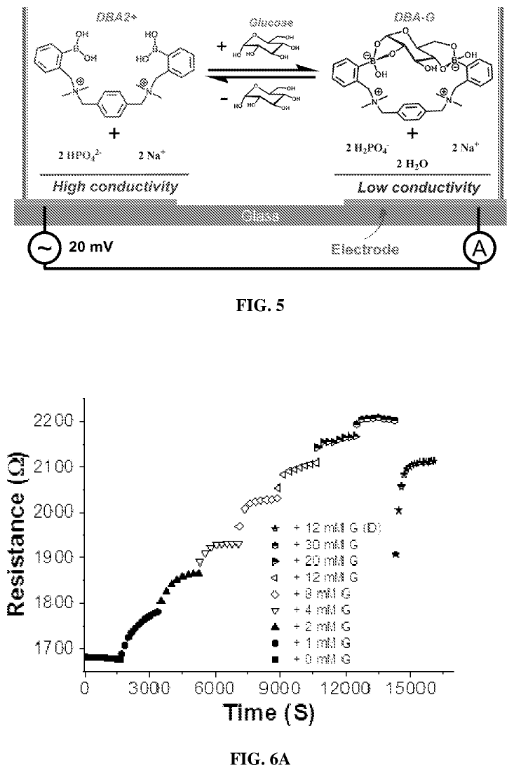

The conductivity sensor is based on the change of the conductivity of ion species in a solution upon diboronic acid compounds binding with glucose. For example, the diboronic acid compounds of any of Formulae I-IV, such as a diboronic acid compound of Formula III can bind glucose, resulting a change of the pKa of the diboronic acid compounds from 9.4 to 6.3 at physiological pH (i.e. pH 7.4), leading to deprotonation. In phosphate buffer solution, the released protons are neutralized by HPO₄²⁻. Thus, glucose mediates the conversion of DBA2+ and HPO₄²⁻ (higher ionic conductivity) to DBA2+/glucose complex (DBA-G) and H₂PO₄⁻ (lower ionic conductivity) (FIG. 5). Typically, the ionic conductivity is measured by solution resistance upon applying a voltage at the electrodes at a frequency.

An exemplary method of using the conductivity sensor for testing the presence, the absence, and/or the concentration of glucose in a biological sample includes: (a) applying a voltage at a frequency; (b) measuring a first resistance of a buffer solution; (c) transferring the biological sample to the buffer solution to form a test sample; and (d) measuring a second resistance of the test sample. Step (b) may be performed simultaneously with, substantially simultaneously with, or subsequent to step (a). Step (d) may be performed simultaneously with, substantially simultaneously with, or subsequent to step (c). Optionally, steps (c) and (d) are repeated two or more times.

For conductivity sensors which include the diboronic acid compound(s) and buffer salt(s) in a solid form in the sample reservoir, the above described exemplary method can be modified to include a step of adding a solvent, preferably water or an aqueous solvent to the reservoir to form a buffer solution, prior to the other steps, particularly prior to step (b).

The biological sample is typically a bodily fluid containing glucose. The biological sample may be transferred from the subject's body and into the buffer solution of the conductivity sensor by any suitable means. For example, the conductivity sensor is placed over the skin site that has been treated by abrasion and the bodily fluid transfers by passive diffusion out of the patient's body and into the buffer solution of the sensor.

Optionally, the voltage is between about 1 mV and about 20 mV, preferably about 20 mV. Impedance spectra in the 1 kHz to 1 MHz range are generally dominated by the sum of the mobilities of individual ionic species. In some embodiments, the frequency is between about 1 kHz and about 1 MHz, preferably about 10⁵ Hz. In some embodiments, the voltage is applied at about 20 mV and the frequency is about 10⁵ Hz.

The second resistance may be lower or higher than the first resistance. In some forms, the difference between the first resistance and the second resistance is a function of glucose concentration. For example, the second resistance is lower than the first resistance and the difference between the first resistance and the second resistance is indicative of glucose concentration.

Further, any difference between the first resistance and second resistance in response to an interference sugar, such as fructose, galactose, maltose, sucrose, and/or lactose, is less than about 3% as compared to the difference between the first resistance and second resistance in response to glucose.

B. Optical Sensor

The optical sensor is based on the change of an optical property of a diboronic acid compound-dye (DBA-D) complex in the presence of glucose, such as absorbance, fluorescence, or a combination of absorbance and fluorescence. For example, the diboronic acid compounds of any one of Formulae I-IV, such as a diboronic acid compound of Formula III, can form a complex (DBA-D) with a dye, such as Alizarin Red S (ARS). The diboronic acid compound favors the formation of diboronic acid compound-glucose (DBA-G) complex in the presence of glucose such that glucose can replace the dye in the DBA-D complex, resulting in a change of the absorbance and/or fluorescence signal (see, e.g., FIGS. 8B and 8C).

An exemplary method of using the optical sensor for testing the presence, the absence, or the concentration of glucose in a biological sample includes: (a) measuring a first absorbance or a first fluorescence of the DBA-D complex; (b) transferring the biological sample into the optical sensor such that the biological sample is in contact with the DBA-D complex; and (c) measuring a second absorbance or a second fluorescence of the DBA-D complex. Step (c) may be performed simultaneously with, substantially simultaneously with, or subsequent to step (b).

The biological sample is typically a bodily fluid containing glucose. The biological sample may be transferred from the subject's body and to the optical sensor and contacts the DBA-D complex by any suitable means. For example, the optical sensor is implanted under the skin and in the bodily fluid of the subject such that the bodily fluid directly flows into the sensor and contacts the DBA-D complex in the sensor. Alternatively, the optical sensor is placed over the skin site that has been treated by abrasion and the bodily fluid transfers by passive diffusion out of the patient's body and into the sensor and contacts the DBA-D complex in the sensor.

The absorbance or fluorescence of the DBA-D complex increases or decreases upon the addition of the sample as a function of glucose concentration (i.e. the second fluorescence is higher or lower than the first fluorescence or the second absorbance is higher or lower than the first absorbance). In some embodiments, the absorbance or fluorescence of the DBA-D complex increases upon the addition of the biological sample containing glucose. In some embodiments, the absorbance or fluorescence of the DBA-D complex decreases upon the addition of the biological sample containing glucose.

Optionally, the exemplary method of using the optical sensor includes a step of adding a buffer solution into the optical sensor that dissolves the DBA-D complex performed prior to step (a).

C. Dual-Mode Sensors

Optionally, sensors that use the disclosed diboronic acid compounds for glucose sensing are dual-mode sensors. A “dual-mode sensor” generally refers to a sensor that measures two types of signals, such as absorbance and fluorescence, absorbance and conductivity, fluorescence and conductivity, absorbance and current, fluorescence and current, etc. For example, a dual-mode optical sensor measures the absorbance and fluorescence signals of the DBA-D complex(es).

An exemplary method of using a dual-mode optical sensor for testing the presence, the absence, or the concentration of glucose in a test sample includes: (a) adding the test sample to the dual-mode optical sensor; and (b) measuring an absorbance or a fluorescence of the DBA-D complex. Step (b) may be performed simultaneously with, substantially simultaneously with, or subsequent to step (a). Typically, the test sample dissolves the DBA-D complex. Optionally, the exemplary method of using the dual-mode optical sensor includes a step of adding a buffer solution into the sensor that is performed prior to step (a) and the test sample is added into the buffer solution.

1. Improving Measurement Accuracy

Optionally, the dual-mode sensor performs self-calibration to determine the glucose concentration in a test sample such that the measurement accuracy is improved compared with a sensor without self-calibration.

For example, existing optical glucose sensors often suffer from photo bleaching of the dye, which leads to decrease of dye concentrations from its initial value during sensor operation. These sensors use a universal calibration curve by plotting a single type of signal vs. standard glucose concentration based on the initial dye concentration to calculate glucose concentration in the test sample, which causes errors unless calibrated. In currently available CGMS, such a change in dye concentration causes drift of test results over time. In contrast, self-calibration allows the sensor to fit two types of signals in a series of calibration curves generated from the same two types of signals in standard solutions and select the closest fitting calibration curve based on the actual concentrations of the DBA and dye of a measurement during continuous sensor operation to calculate glucose concentration, thereby improve the accuracy of measurement compared with a measurement using sensors without calibration.

A process of self-calibration is described below. “Accuracy of measurement” generally refers to the difference between a glucose concentration measured using the optical sensor and the glucose concentration measured using a standard method, such as YSI measurement, from the same test sample. An exemplary standard method is described in the YSI-2900-Series-Manual.

Additionally, self-calibration can reduce the frequency of calibration compared to sensors without self-calibration. For example, to avoid errors, sensors without self-calibration may require daily calibration, such as Eversense, which generally requires two calibrations per day. Optical sensors with self-calibration can be used continuously for at least 7 days without calibration, at least 10 days without calibration, at least 14 days without calibration, at least 30 days without calibration, at least 45 days without calibration, at least 60 days without calibration, or at least 90 days without calibration.

2. Self-Calibration

“Self-calibration” as used herein refers to the process of fitting values of two types of signals measured in a test sample with calibration curves generated from the same two types of signals and selecting the closest fitting calibration curve for the calculation of glucose concentration in the test sample.

a. Establishing Calibration Curves

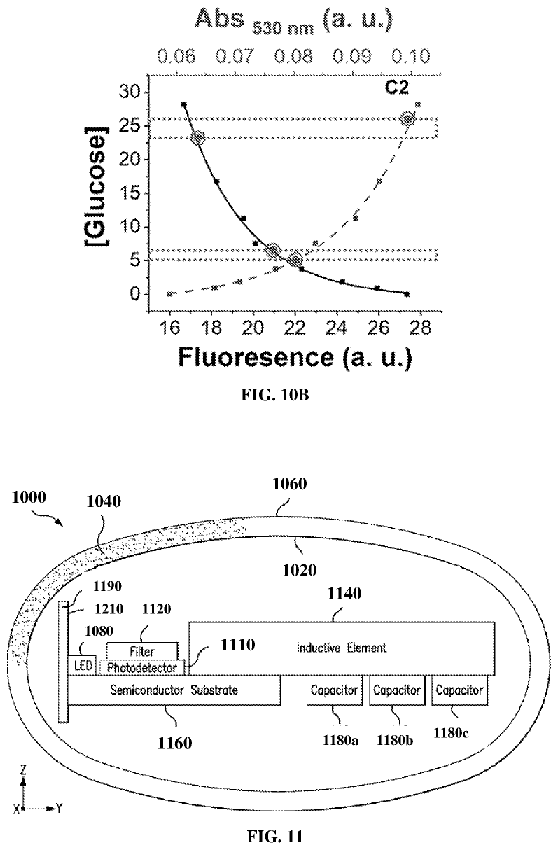

The calibration curves are generated using values of the same two types of signals measured from standard solutions. For example, a first series of standard solutions containing a DBA-D complex at a first fixed diboronic acid compound (DBA) concentration and a first fixed dye (D) concentration, and glucose in a range of concentrations are measured to generate a first set of absorbance values and fluorescence values. These absorbance values are plotted against fluorescence values to produce a first calibration curve (see, e.g., FIG. 10A). A second series of standard solutions containing the same DBA-D complex at a second fixed DBA concentration and a second fixed D concentration, and glucose in the same range of concentrations are measured to generate a second set of absorbance values and fluorescence values. These absorbance values are plotted against fluorescence values to produce a second calibration curve.

The first fixed concentration of the DBA may be the same, substantially the same, or different from the second fixed concentration of the same DBA and the first fixed D concentration may be the same, substantially the same, or different from the second fixed D concentration, as long as the concentration ratio of DBA:D in the first series of standard solutions is different from that in the second series of standard solutions.

Optionally, more than two calibration curves are produced following this procedure, for example, at least 3 calibration curves, at least 4 calibration curves, at least 5 calibration curves, at least 6 calibration curves, at least 7 calibration curves, at least 8 calibration curves, at least 9 calibration curves, at least 10 calibration curves, at least 15 calibration curves, at least 20 calibration curves, at least 25 calibration curves, at least 30 calibration curves, at least 35 calibration curves, at least 40 calibration curves, at least 45 calibration curves, at least 50 calibration curves, at least 55 calibration curves, at least 60 calibration curves, at least 65 calibration curves, at least 70 calibration curves, at least 75 calibration curves, at least 80 calibration curves, at least 85 calibration curves, at least 90 calibration curves, at least 95 calibration curves, or at least 100 calibration curves can be produced, where each calibration curve represents a specific combination of DBA concentration and D concentration.

b. Fitting of Measured Signals

A test sample in which the concentration of glucose is unknown is measured using the same DBA-D complex at fixed DBA and D concentrations to generate an absorbance value and a fluorescence value.

The absorbance value and fluorescence value measured from the test sample are then fitted with the calibration curves generated from standard solutions. The closest fitting calibration curve is selected for the calculation of the glucose concentration in the test sample (see, for example, FIG. 10A).

c. Calculating Glucose Concentrations

The concentration of glucose in the test sample is calculated based on the selected calibration curve. Typically, the concentration of glucose is calculated by fitting the measured absorbance and/or fluorescence values of the test sample in a first and/or a second calculation curve. The first and second calculation curves can be generated by plotting glucose concentrations as a function of absorbance using data of the selected calibration curve (i.e. first calculation curve) and plotting glucose concentrations as a function of fluorescence using data of the selected calibration curve (i.e. second calculation curve). See, for example, FIG. 10B.

d. Exemplary Self-Calibration Steps

Self-calibration of the sensor may be performed by a processor in the sensor. Generally, when self-calibration is applied, the sensor can determine the concentration of glucose in a test sample by (i) fitting the measured absorbance and fluorescence of the test sample with pre-established calibration curves, (ii) selecting the closest fitting calibration curve, and (iii) calculating the concentration of glucose in the test sample based on data of the selected calibration curve. See, for example, FIGS. 10A-10B. D. Continuous Glucose Monitoring System

The conductivity sensors and/or optical sensors can be used in a continuous glucose monitoring system (CGMS) as described above.

1. Applying the CGMS

Typically, the CGMS applied on a skin site or implanted under the skin of a subject, such as a human. Following application, the CGMS extracts or is in direct contact with the interstitial fluid and/or blood from the subject. Optionally, one or more attachment means are used to secure the CGMS to the abraded skin site. A variety of attachment means may be used, including, but not limited to, adhesive, straps, and elastic bands/chords.

The CGMS is configured to continuously and accurately measure glucose level over a time period of at least 7 days without calibration, at least 10 days without calibration, at least 14 days without calibration, at least 30 days without calibration, at least 45 days without calibration, at least 60 days without calibration, or at least 90 days without calibration.

a. Implanting the CGMS Under the Skin

Optionally, the CGMS is implanted under the skin of the subject by a user, such as a medical professional, such that the sensor is in direct contact with the interstitial fluid and/or blood of the mammal. Methods for implanting the CGMS are known in the art. For example, a medical professional makes a small incision, places the sensor under the skin at a body part (e.g. arm) of the subject, and closes the incision, such as with steri strips.

b. Applying the CGMS on a Skin Site

i. Penetrating Skin Using Microneedles

Optionally, the CGMS includes a component for extracting interstitial fluid from the subject (e.g. a plurality of microneedles) and is applied on a skin site of the subject by a medical professional or the subject being tested (i.e. self-application). Typically, the medical professional or the subject presses the CGMS against the skin of the subject such that the microneedles penetrate the skin and thus can extract interstitial fluid and/or blood from the subject to the sensors included in the CGMS.

ii. Abrading or Permeabilizing the Skin

Alternatively, the CGMS is applied on a permeabilized skin site of the subject by a medical professional or the subject being tested (i.e. self-application), such as one which has been abraded or permeabilized by iontophoresis, sonophresis, or by applying permeation enhancing agents to the skin site.

Prior to applying the CGMS to the site on the subject's skin, the permeability of the skin site is increased. Optionally, the stratum corneum is removed in a controlled manner. Any suitable permeabilization device and method may be used to increase the permeability of the skin site. Typical methods for increasing the skin's permeability include abrasion, tape stripping, rubbing, sanding, laser ablation, radio frequency (RF) ablation, chemicals, sonophoresis, iontophoresis, electroporation, application of permeation enhancing agents. Optionally, permeability of the skin is increased to the desired level using a controlled skin abrasion device.

Optionally, the permeabilization step is continued until the desired permeability level is achieved, which can be determined by measuring its transepidermal water loss (TEWL). The TEWL can be determined using technologies from cyberDERM Inc. or Delfin Technologies (such as the Vapometer). Optionally, following the permeabilization step, the skin site has a TEWL of between about 20 to 50 g/m²/hr or between about 30 to 40 g/m²/hr.

2. Transfer of Biological Samples to the CGMS

Bodily fluid containing glucose may transfer from the subject's body and into the CGMS by any suitable means. For example, the CGMS is implanted under the skin and in the bodily fluid of the subject such that glucose in the bodily fluid directly flows into the sensor of the CGMS.

Optionally, the CGMS is placed over the skin site that has been treated by abrasion and the bodily fluid transfers by passive diffusion out of the patient's body and into the CGMS.

Alternatively, the CGMS contains microneedles and pressed on the skin. The bodily fluid transfers from the subjects' body and into the CGMS by capillary forces through the microneedles of the CGMS.

3. Analysis of Signals

Generally, a first signal is generated prior to applying the CGMS on a skin site of the subject or implanting the CGMS under the skin of the subject. After CGMS application or implantation, glucose in the bodily fluid is transferred into the CGMS and in contact with the diboronic acid compound(s) in the CGMS, generating a second signal. The processor subtracts the first signal from the second signal and determines a differential signal, which corresponds with at least one glucose level data point.

For example, a first conductivity signal is generated prior to applying the CGMS that contains a conductivity sensor on a skin site of the subject. After CGMS application, glucose in the bodily fluid is extracted out of the subject's body and into the CGMS and binds with the diboronic acid compound in the CGMS and generates a second conductivity signal. The processor subtracts the first conductivity signal from the second conductivity signal and determines a differential conductivity signal, which corresponds with a concentration of glucose.

Alternatively, a first absorbance signal or a first fluorescence signal is generated prior to implanting the CGMS that contains an optical sensor under the skin of the subject. After CGMS implantation, glucose in the bodily fluid flows into the CGMS and binds with the diboronic acid compound of the DBS-D complex to replace the dye and generates a second absorbance signal or a second fluorescence signal. The processor subtracts the first absorbance signal from the second absorbance signal or the first fluorescence signal from the second fluorescence signal and determines a differential absorbance or fluorescence signal, which corresponds with a concentration of glucose.

Optionally, the CGMS contains a dual mode sensor that measures two types of signals and performs self-calibration. For example, when using a CGMS containing a dual mode sensor, calibration curves are established by plotting a plurality of absorbance signals vs a plurality of fluorescence signals measured in standard solutions as described above and the data is stored in the processor of the CGMS. After CGMS application or implantation, glucose in the bodily fluid transfers into the CGMS and binds with the diboronic acid compound of the DBS-D complex to replace the dye and generates a measured absorbance signal and a measured fluorescence signal. The processor then performs the self-calibration as described above to determine the concentration of glucose.

The disclosed diboronic acid compounds, sensors, and methods can be further understood through the following numbered paragraphs.

1. A diboronic acid compound having a structure of Formula I:

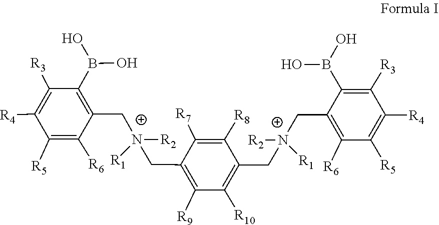

wherein R₁ and R₂ are independently an unsubstituted alkyl group, a substituted alkyl group, an unsubstituted heteroalkyl group, or a substituted heteroalkyl group; and
wherein R₃-R₁₀ are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonate ester group containing one substituent, an ether group containing one substituent, an aminooxy group optionally containing one or two substituents at the amino nitrogen, or a hydroxyamino group optionally containing one or two substituents,

wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

2. The compound of paragraph 1, wherein R₁ and R₂ are independently unsubstituted or substituted alkyl groups, preferably unsubstituted or substituted C₁-C₁₀ alkyl groups, more preferably unsubstituted or substituted linear C₁-C₁₀ alkyl groups, most preferably unsubstituted or substituted methyl groups having a structure of Formula II:

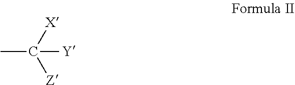

wherein X′, Y′, and Z′ are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonate ester group containing one substituent, an ether group containing one substituent, an aminooxy group optionally containing one or two substituents at the amino nitrogen, or a hydroxyamino group optionally containing one or two substituents,

wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

3. The compound of paragraph 2, wherein X′, Y′, and Z′ are independently a hydrogen, a halogen atom, a nitrile group, a methyl group, or an unsubstituted aryl group.

4. The compound of any one of paragraphs 1-3, having a structure of Formula III:

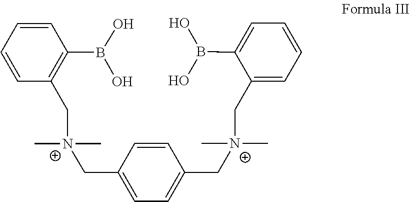 [SMILES: C[N+](C)(Cc1ccc(C[N+](C)(C)Cc2ccccc2B(O)O)cc1)Cc1ccccc1B(O)O]

5. A diboronic acid compound having a structure of Formula IV:

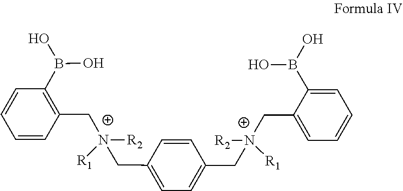

wherein R₁ and R₂ are independently an unsubstituted alkyl group, a substituted alkyl group, an unsubstituted heteroalkyl group, or a substituted heteroalkyl group, preferably an unsubstituted alkyl group or a substituted alkyl group, more preferably an unsubstituted C₁-C₁₀ alkyl group or a substituted C₁-C₁₀ alkyl group.

6. The compound of any one of paragraphs 1-5 further comprising counter ions to the tertiary amine groups.

7. The compound of paragraph 6, wherein the counter ions are halide anions, phosphate ion, hydrogen phosphate ion, dihydrogen phosphate ion, trihydrogen phosphate ion, or bicarbonate, or a combination thereof.

8. The compound of paragraph 6 or paragraph 7, wherein the counter ions are dihydrogen phosphate ions.

9. The compound of any one of paragraphs 1-8, wherein the compound has a solubility of at least 1 g/L in aqueous solution at pH 7.4 and 25° C.

10. The compound of any one of paragraphs 1-9, wherein the compound binds glucose with a K_{d} value between about 0.1 mM and about 30 mM.

11. The compound of any one of paragraphs 1-10, wherein the compound binds glucose with a K_{d} value at least about 2-times lower, at least about 5-times lower, at least about 10-times lower, at least about 15-times lower, or at least about 20-times lower than a K_{d} value for an interference sugar under the same conditions.

12. The compound of paragraph 11, wherein the interference sugar is selected from the group consisting of fructose, galactose, maltose, sucrose, and lactose, or a combination thereof.

13. The compound of any one of paragraphs 1-12 having a pKa value between about 7.4 and about 10.5, preferably between about 8.5 and about 10.5, more preferably between about 9 and about 10. 14. The compound of paragraph 13, wherein the pKa value increases or decreases upon binding with glucose.

15. The compound of paragraph 13 or paragraph 14, wherein the pKa value increases or decreases by about 1 unit, about 2 units, preferably about 3 units, more preferably about 4 units upon binding with glucose.

16. The compound of any one of paragraphs 13-15, wherein the pKa value decreases by about 1 unit, about 2 units, preferably about 3 units, more preferably about 4 units upon binding with glucose.

17. A conductivity sensor for measuring glucose concentration in a biological sample comprising

a reservoir comprising the compound of any one of paragraphs 1-16 and a buffer solution;

a pair of electrodes; and

a membrane,

wherein the electrodes are in electrical communication with each other,

wherein an electrically conductive surface of each electrode is in contact with the buffer solution, and

wherein the membrane is configured to prevent or reduce ion exchange between the buffer solution and the biological sample.

18. A conductivity sensor for measuring glucose concentration in a biological sample comprising

a reservoir comprising the compound of any one of paragraphs 1-16 and buffer salts therein;

a pair of electrodes; and

a membrane,

wherein the electrodes are in electrical communication with each other,

wherein the compound and the buffer salts are in the form of a solid, optionally in the form of a powder, and

wherein an electrically conductive surface of each electrode is in contact with the opening of the reservoir.

19. The conductivity sensor of paragraph 17 or 18, wherein the reservoir is defined by side walls and a bottom surface, and contains an opening configured to allow the biological sample to enter the reservoir, optionally wherein an electrically conductive surface of each electrode is part of or forms one or more of the side walls and/or bottom surface of the reservoir.

20. The conductivity sensor of any one of paragraphs 17-19, wherein the membrane is located adjacent to the opening of the reservoir, and defines an outer surface that encloses the buffer solution or solid buffer salts and compound inside of the reservoir.

21. The conductivity sensor of any one of paragraphs 17-20, wherein the membrane is a bipolar membrane

22. The conductivity sensor of any one of paragraphs 17-21, further comprising a detector.

23. A method of testing the presence, absence, and/or the concentration of glucose in a biological sample using the conductivity sensor of any one of paragraphs 17-22 comprising:

(a) applying a voltage at a frequency;

(b) measuring a first resistance of the buffer solution;

(c) transferring the biological sample into the reservoir to combine with the buffer solution and form a test sample; and

(d) measuring a second resistance of the test sample,

wherein step (b) is performed simultaneously with, substantially simultaneously with, or subsequent to step (a), and

wherein step (d) is performed simultaneously with, substantially simultaneously with, or subsequent to step (c).

24. A method of testing the presence, absence, and/or the concentration of glucose in a biological sample using the conductivity sensor of any one of paragraphs 18-22 comprising:

(a) adding a solvent, preferably water or an aqueous solvent to the reservoir to form a buffer solution,

(b) applying a voltage at a frequency;

(c) measuring a first resistance of the buffer solution;

(d) transferring the biological sample into the reservoir to combine with the buffer solution and form a test sample; and

(e) measuring a second resistance of the test sample,

wherein step (b) is performed simultaneously with, substantially simultaneously with, or subsequent to step (a), and

wherein step (d) is performed simultaneously with, substantially simultaneously with, or subsequent to step (c).

25. The method of paragraph 23 or paragraph 24 further comprising repeating steps (c) and (d).

26. The method of any one of paragraphs 23-25, wherein the voltage is between about 1 mV and about 20 mV, preferably about 20 mV.

27. The method of any one of paragraphs 23-26, wherein the frequency is between about 1 kHz and about 1 MHz, preferably about 10⁵ Hz.

28. An optical sensor comprising the compound of any one of paragraphs 1-16, a dye, a light source, and a detector

wherein the compound and the dye form a complex (DBA-D complex).

29. The optical sensor of paragraph 28, further comprising a processor, a transmitter, or an output display, or a combination thereof.

30. A method of testing the presence, the absence, and/or the concentration of glucose in a biological sample using the optical sensor of paragraph 28 or paragraph 29 comprising:

(a) measuring a first fluorescence or a first absorbance of the DBA-D complex;

(b) transferring the biological sample to the optical sensor such that the biological sample is in contact with the DBA-D complex; and

(c) measuring a second fluorescence or a second absorbance of the DBA-D complex,

wherein step (c) is performed simultaneously with, substantially simultaneously with, or subsequent to step (b).

31. The method of paragraph 30, further comprising (d) adding a buffer solution into the optical sensor that dissolves the DBA-D complex, wherein step (d) is performed prior to step (a).

32. The method of paragraph 30 or paragraph 31, further comprising repeating steps (b) and (c) two or more times.

33. A method of testing the presence, the absence, and/or the concentration of glucose in a biological sample using the optical sensor of paragraph 28 or paragraph 29 comprising:

(a) adding the biological sample to the optical sensor such that the biological sample is in contact with the DBA-D complex; and

(b) measuring an absorbance and a fluorescence of the DBA-D complex,

wherein step (b) is performed simultaneously with, substantially simultaneously with, or subsequent to step (a), and

wherein the optical sensor performs self-calibration to determine the concentration of glucose in the test sample.

34. A continuous glucose monitoring system (CGMS) comprising:

(a) a conductivity sensor of any one of paragraphs 17-22 or an optical sensor of paragraph 28 or paragraph 29; and optionally

(b) a bipolar membrane; and/or

(c) a microneedle, optionally an array of microneedles for fluid extraction.

35. The continuous glucose monitoring system of paragraph 34 comprising two or more of the conductivity sensors or two or more of the optical sensors.

36. A method of monitoring glucose level in a subject using the CGMS of paragraph 34 or paragraph 35 comprising

(a) applying the CGMS on a skin site of the subject or implanting the CGMS under the skin of the subject.

37. The method of paragraph 36, comprising (a) applying the CGMS on a skin site of the subject, and further comprising permeabilizing the skin site of the subject prior to step (a).

The present invention will be further understood by reference to the following non-limiting examples.

### EXAMPLES

### Example 1. Synthesis of DBA2+

Materials and Methods

Materials and Instruments

Solvents and materials were purchased from suppliers (Fisher Scientific, Sigma Aldrich and Acros) and were used without further purification. ¹H and ¹³C nuclear magnetic resonance (NMR) spectra were obtained on a Varian 500 MHz spectrometer, and ³¹P NMR spectra were obtained on a Varian 400 MHz spectrometer. Ultraviolet-visible absorption spectra and absorbance at 280 nm were recorded on microplate readers (Tecan M220 Infinite Pro or Tecan Spark 10M). Electrochemical impedance spectra and solution resistance were measured on a Solartron 1260 Impedance Analyzer.

Synthesis of DBA2+Br

Dimethyl amine (60 mL, 2 M in tetrahydrofuran (THF)) was cooled down to −78° C. for 10 min in dry ice/acetone bath. 1,4-dibromomethyl benzene (5.3 g, 20 mmol) dissolved in 20 mL THF was slowly added. Then the solution was warmed to room temperature. After 1 h reaction, the reaction solution was poured to a mixture of 400 mL of ethyl acetate and 100 mL of 1 M K₂CO₃ aqueous solution. After vigorous stirring and phase separation, the organic phase was collected and dried over Na₂SO₄. The crude intermediate product, 1,4-bis(dimethylaminomethyl) benzene, was obtained with a yield of 95% by removing solvent under rotary evaporation. Without any further purification, 1,4-bis(dimethylaminomethyl) benzene was dissolved in 40 mL of anhydrous DMF together with 2-bromomethylphenyl boronic acid (12.9 g, 60 mmol). After bubbling with argon for 20 min, the reaction was heated to 60° C. and kept for 24 h. The reaction mixture was then precipitated in 200 mL of ethyl acetate, and the sediment was washed with 20 mL of ethyl acetate twice and dried under vacuum. The residue was purified by C18-reversed phase silica gel chromatography using water/methanol=9:1 as eluent and afford a white solid after removing methanol and lyophilization (3.8 g, 30%).

¹H NMR (500 MHz, DMSO-d6, δ): 8.37 (s, 4H), 7.4-7.8 (m, 12H), 4.89 (s, 4H), 4.80 (s, 4H), 2.90 (s, 12H).

¹³C NMR (125 MHz, CDCl₃, δ): 140.03, 135.13, 134.51, 134.03, 131.66, 130.52, 130.00, 129.73, 67.50, 66.35, 48.82. HRMS (ESI) m/z: (M-2Br⁻)²⁺ calcd. 231.1425; found 231.1432.

Synthesis of DBA2+P

DBA2+Br (310 mg, 0.5 mmol) was mixed with 3 g of C18-reversed phase silica, and then dry-load on C18-reversed phase silica gel column. After flushing with 200 mM NaH₂PO₄ aqueous solution (about 2 column volume (CV)) to wash out the bromide anion and 10 μM H₃PO₄ solution for another 2 CV to wash out the NaH₂PO₄, 10 μM H₃PO₄ solution/methanol=9:1 was then used as eluent to afford a white solid after removing methanol and lyophilization (200 mg, 60%).

The anion change was verified by ¹H NMR and ³¹P NMR spectra of mixture of DBA2+P and tetrabutylammonium hexafluorophosphate (TBAHFP) in deuterated methanol by comparing the ratios of proton (1.2) and phosphor integration, respectively.

¹H NMR (400 MHz, Methanol-d4, δ): DBA2+P: 7.5-7.9 (m, 12H), 4.7-4.8 (d, 8H), 2.96 (s, 12H); TBAHFP: 3.23 (t, 9.6H, 1.2 eq), 1.65 (t, 9.6H, 1.2 eq.), 1.40 (t, 9.6H, 1.2 eq.), 1.02 (t, 14.3H, 1.2 eq.).

³¹P NMR (162 MHz, Methanol-d4, δ): DBA2+P: 1.20 (s, 2P); TBAHFP: −154 to −136 (m, 1.3P, 1.3 eq.). HRMS (ESI) m/z: (M-2H₂PO₄⁻)²⁺ calcd. 231.1425; found 231.1429.

Results

A scheme of the synthesis of DBA2+Br is:

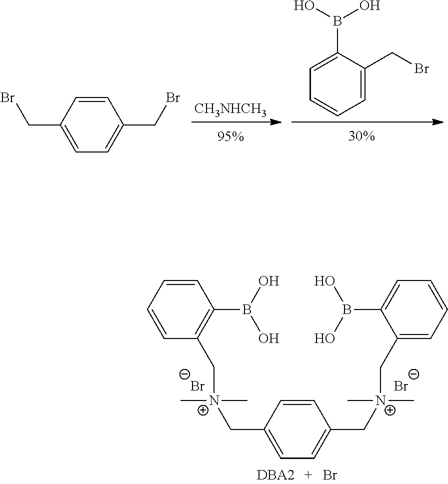

Scheme of the replacement of counter anion bromide with phosphate:

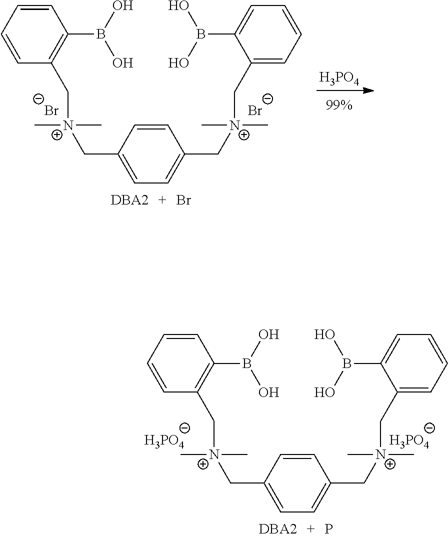

The schemes above show the remarkably simple synthesis of DBA2+.

### Example 2. DBA2+ Shows a Change of pKa Upon Glucose Binding in an Aqueous Medium at about pH 7.4

Materials and Methods

pKa Measurements

100 mM buffer solutions for titration from pH 4 to pH 11.5 were prepared with different buffer systems to ensure the accuracy of pH for the following tests. NaAc/HAc was prepared for pH 4.05, 4.53, 5.03 and 5.53; Na₂HPO₄/NaH₂PO₄ was prepared for pH 6.00, 6.51, 6.99 and 7.51; NaB(OH)₄/B(OH)₃ was prepared for pH 8.05, 8.56 9.03 and 9.53; Na2CO3/NaHCO3 was prepared for pH 10.01, 10.54, 11.01 and 11.49.

50 μL of buffer solutions at different pH was added into 96 microplate wells. Then 50 μL of 2 mM DBA2+Br or a mixture of 2 mM DBA2+Br and 400 mM glucose in water was added to each well.

After shaking for 10 s and waiting for 10 min, the absorbance of each well at 280 nm was recorded on a microplate reader.

pKa Calculations

Due to the interaction between borate and glucose, the pH of the borate buffer solution is changed and thus the data for DBA2+Br/Glucose from pH 8 to 9.5 were abandoned. The rest of the data was plotted in Origin. The equilibrium equation for DBA2+ is as follows:

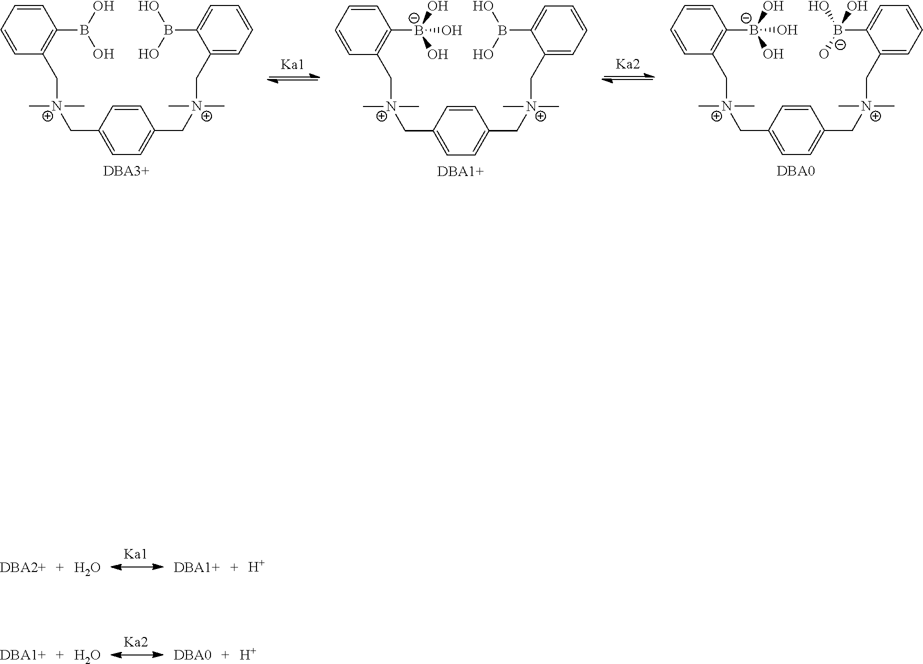

Thus, the first and second acid dissociation constants, Ka1 and Ka2, are:

$$Ka 1 = [ DBA 1 + ] \times [ H + ] / [ DBA 2 + ] \\ K a 2 = [ DBA 0 ] \times [ H + ] / [ DBA 1 + ] \qquad ( 1 )$$

According to the law of conservation of mass, total concentration of diboronic acid compounds, C, can be expressed as:

$$C = [ DBA 2 + ] + [ DBA 1 + ] + [ DBA 0 ] \qquad ( 2 )$$

Assuming that the molar extinction coefficient at 280 nm of neutral phenylboronic acid is E, and that for the anionic type it is ε+Δε, the absorbance of the solution Abs at 280 nm is:

$$Abs = 2 \times ε \times [ DBA 2 + ] + ε \times [ DBA 1 + ] + ( ε + Δε ) \times [ DBA 1 + ] + 2 \times ( ε + Δε ) \times [ DBA 0 ] = 2 εC + Δε \times ( [ DBA 1 + ] + 2 [ DBA 0 ] ) \qquad ( 3 )$$

From equations (1), (2) and (3), equation (4) is obtained:

$$Abs = 2 ε C + Δε \times C \times \frac{K a 1 \times [ \text{?} ] + 2 \times K a 1 \times Ka 2}{{[ \text{?} ]}^{2} + K a 1 \times [ \text{?} ] + K a 1 \times K a \text{?}} \\ \text{?} \text{indicates text missing or illegible when filed} \qquad ( 4 )$$

Then equation (4) is used for non-linear fitting in Origin, and pKa is defined as the pH at which half of the boronic acid is in the anionic state:

$$pKa = - log ( Ka 1 \times K a 2 ) \qquad ( 5 )$$

The same non-linear curve fitting also was used for the determination of pKa of DBAG2+G which has equilibrium equation as follow:

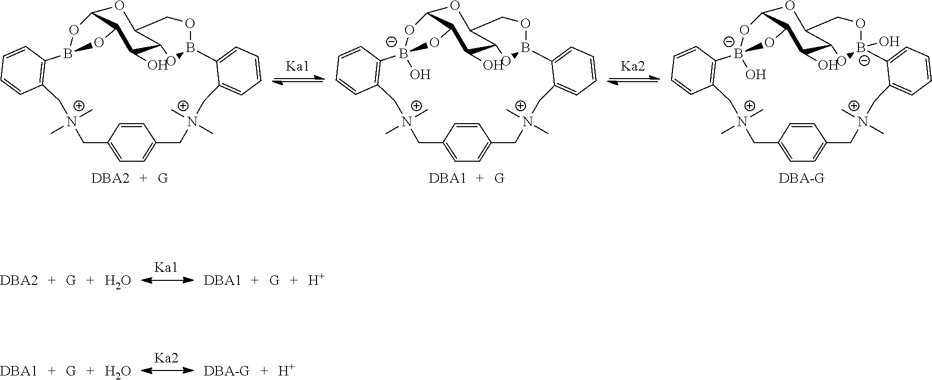

Results

The pKa of DBA2+ before and after glucose binding was determined based on the differences in absorption spectra upon formation of tetrahedral borate anion in high pH media (Springsteen, et al., Tetrahedron, 58:5291-5300 (2002)).

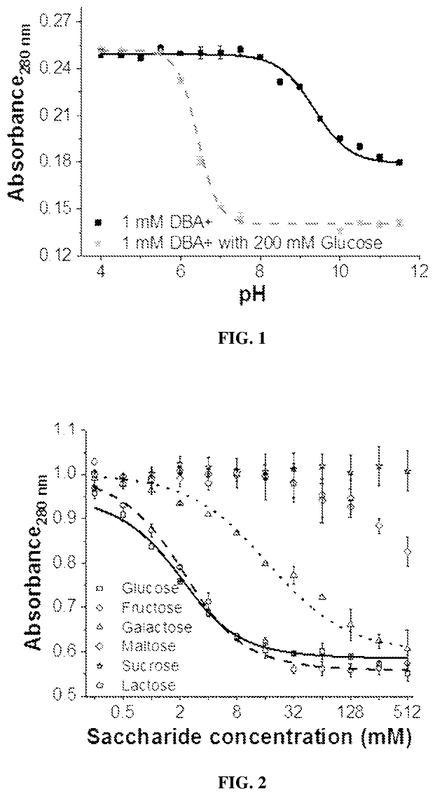

pKa values were determined by curve fitting the changes in absorbance as a function of pH (FIG. 1).

DBA2+ exhibits a pKa value of 9.4.

In the presence of 200 mM glucose, conditions that provide DBA-G, one observes a shift in the plot, from which one derives a pKa value of 6.3.

The ˜3 unit change in pKa for DBA2+ and DBA-G centers around pH=7.4. These conditions provide the basis for the response toward glucose under physiologically relevant conditions.

### Example 3. DBA2+ Shows High Selectivity to Glucose Compared to Interference Sugars

Materials and Methods

Disassociation Constant (Kd) Measurements

100 μL of glucose aqueous solutions with two-fold serial dilutions from 1024 mM to 0.5 mM were added into a 96 well microplate. Then 100 μL of 2 mM DBA2+Br in 100 mM phosphate buffer (pH=7.4) was added. After shaking for 10 s and waiting for 30 min, the absorbance of each well at 280 nm was recorded on a microplate reader.

The affinity and selectivity of DBA+ to glucose were then tested by comparing affinity with other five mono- or di-saccharides, particularly fructose, galactose, maltose, sucrose, and lactose. The UV absorption changes at 280 nm of DBA+ with the increase of sugar concentration was used for the affinity calculation. The absorbance values of 1 mM DBA+Br at 280 nm as function of sugar concentrations from 0 to 512 mM were measured in 50 mM Phosphate PBS (pH=7.4).

Kd Calculations

The titration data was plotted. The disassociation equation for complex DBAG is as follows:

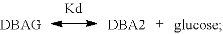

where DBAG refers to all three possible complexes, DBA2+G, DBA1+G and DBA-G, and DBA2 refers to all the unbound molecules, DBA2+, DBA1+ and DBA0.

The disassociation equilibrium rate constant (Kd) is then

$$K \text{?} = \frac{[ \text{?} ] \times [ Glucose ]}{[ DBAG ]} = \frac{( C 1 - [ DBAG ] \text{?} [ C 2 - [ DBAG ] )}{[ DBAG ]} \\ \text{?} \text{indicates text missing or illegible when filed} \qquad ( 6 )$$

Letting C1 be the total concentration of diboronic acids, and C2 be the total concentration of glucose, it is obtained

$${[ DBAG ]}^{2} - ( C 1 + C 2 + Kd ) \times [ DBAG ] + C 1 \times C 2 = 0. \qquad ( 7 )$$

The standard general solution of equation (7) is

$$[ DBAG ] = 0.5 \times ( C 1 + C 2 + Kd - sqrt ( {( C 1 + C 2 + Kd )}^{2} - 4 \times C 1 \times C 2 ) ) , \qquad ( 8 )$$

where sqrt refers to the square root.
The pKa1 and pKa2 for DBA2+ is 9.0 and 9.7, respectively. Thus, at pH 7.4, the concentration of DBA1+ and DBA0 can be ignored. The pKa1 and pKa2 for DBA2+ in the presence of 200 mM glucose is 5.9 and 6.9, respectively. Thus, the concentration ratio of the complexes [DBA-G] to complex [DBA1+G] is 3.16:1, and the concentration of complex DBA2+/G is negligible. Thus, there is

$$C 1 \approx [ DBA 2 ] + [ DBAG ] \qquad ( 9 )$$

$$[ DBAG ] \approx [ DBA 1 + G ] + [ D B A - G ] = 4.16 \times [ DBA 1 + G ] \qquad ( 10 )$$

Assuming the molar extinction coefficient at 280 nm of neutral phenylboronic acid is E, and that for the anionic type it is ε+Δε, the absorbance of the solution Abs at 280 nm is

$$Abs = 2 \times ε \times [ DBA 2 + ] + ε \times [ DBA 1 + G ] + ( ε + Δε ) \times [ DBA 1 + G ] + 2 \times ( ε + Δε ) \times [ DBA - G ] = 2 εC1 + 0.875 \times Δε \times ( C 1 + C 2 + Kd - sqrt ( {( C 1 + C 2 + Kd )}^{2} - 4 \times C 1 \times C 2 ) ) ) , \qquad ( 11 )$$

where sqrt refers to the square root 11 Equation (11) is used for non-linear fitting in Origin to obtain the Kd values.

Results

The disassociation constant (K_{d}) of DBA2+ was calculated through non-linear curve fitting according to previous work (Stootman, et al., Analyst, 131:1145-1151 (2006)).

The K_{d} value for glucose was found to be 0.9±0.1 mM (FIG. 2, square). The K_{d} values for fructose and galactose were determined to be 1.7 mM and 16 mM, respectively (FIG. 2, circle and triangle, respectively). The K_{d} values for maltose, sucrose and lactose could not be determined due to their low affinity towards DBA2+.

DBA2+ therefore showed selectivity to glucose and fructose relative to other saccharides. The maximum physiological or therapeutic plasma concentrations of fructose (0.13 mM) and galactose (0.28 mM) are well below those for glucose (normal range: 4-8 mM, diabetic range 0-30 mM). Due to the marked difference in absolute concentrations, any influence by the presence of fructose and/or galactose on the DBA2+ based system can be negligible (Lorenz, et al., Diabetes Technol. Ther., 20:344-352 (2018)).

### Example 4. DBA2+ Based Conductive Assay Shows Reversible and Reproducible Response to Glucose

Materials and Methods

Electrochemical Impedance Spectra and Solution Resistance Measurements

A conductimetric assay based on solution resistance (R) was developed.

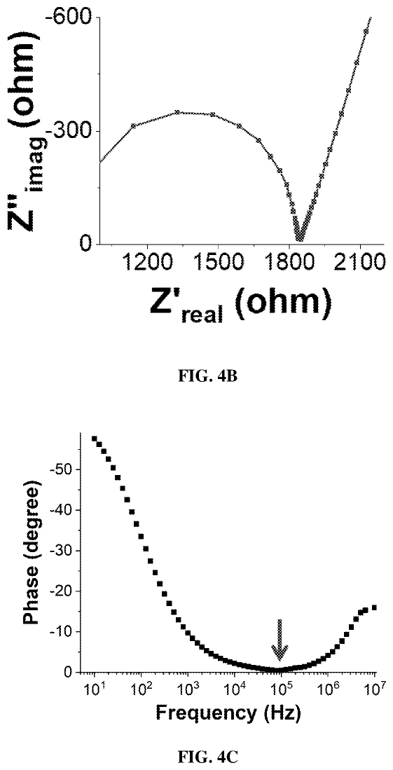

To a reservoir in a device set up as depicted shown in FIG. 3, 1 mL of the initial testing solution (2 mM DBA2+P and 2.5 mM Na3PO4, pH=7.6) was added. Direct current voltage is 0 mV due to the same material for two electrodes. Alternating current voltage was set at 20 mV, and the impedance was scanned vs. frequency from 10 Hz to 10 MHz (FIGS. 4A-4C). At 0.1 MHz, the capacitive reactance is very small compared to the resistance (R) of the solution, and thus this frequency was used for the following tests.

To the testing solution, trace quantities (2-5 μL) of a concentrated (0.5 or 2 M) glucose aqueous solution were continuously added. Glucose solutions were added every 30 minutes with concentrations spanning the diabetes-relevant range, (i.e. [glucose]=0-30 mM). The resistance of solution vs. time was collected for 30 min after each addition. After adding glucose to 30 mM, the testing solution was diluted to 12 mM by adding 1.5 mL of fresh testing solution. 1.5 mL was then discharged after mixing and the resistance of the remaining 1 mL solution was monitored.

For the control, the same volume of water instead of glucose solution was tested.

In the assessment of the reproducibility of the approach, quadruplicate measurements of R were carried out the same as described above but in an incubator at 30° C.

Results

The impedance vs. frequency response was monitored at 20 mV at frequency of 1×10⁵ Hz. These conditions minimize contributions from capacitance and reactance, relative to the resistance (R) (see FIGS. 4A-4C).

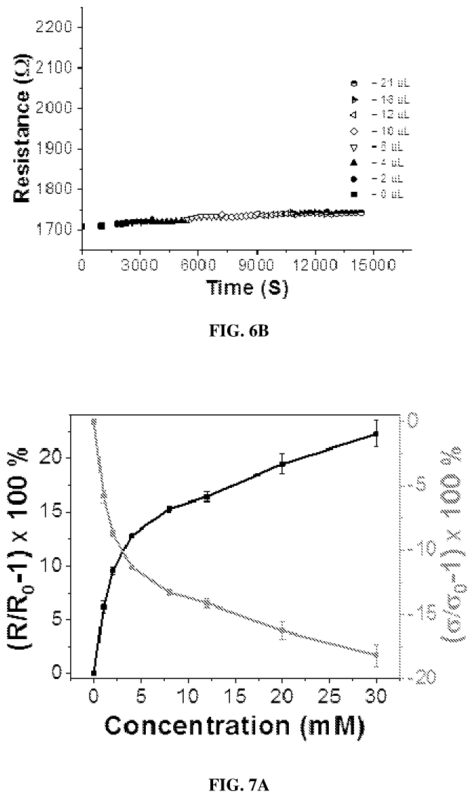

Using the method described above, changes in R of the solution over time as a function of glucose were measured (FIG. 6A). An increase in solution resistance after each glucose addition over the full range of glucose concentrations was observed. In contrast, in the control study, which used water instead of glucose solution, a much narrower range of R values was observed (FIG. 6B). After the addition of the maximum glucose concentration, 1.5 mL of fresh testing solution was added to dilute the glucose concentration from 30 mM to 12 mM, and 1 mL of the mixture was left in the reservoir for continued test. After equilibration, the R value reaches almost the same value (R=2115Ω) as the previous test for 12 mM (R=2109Ω) (see FIG. 6A). This result demonstrates a reversible and repeatable response to glucose.

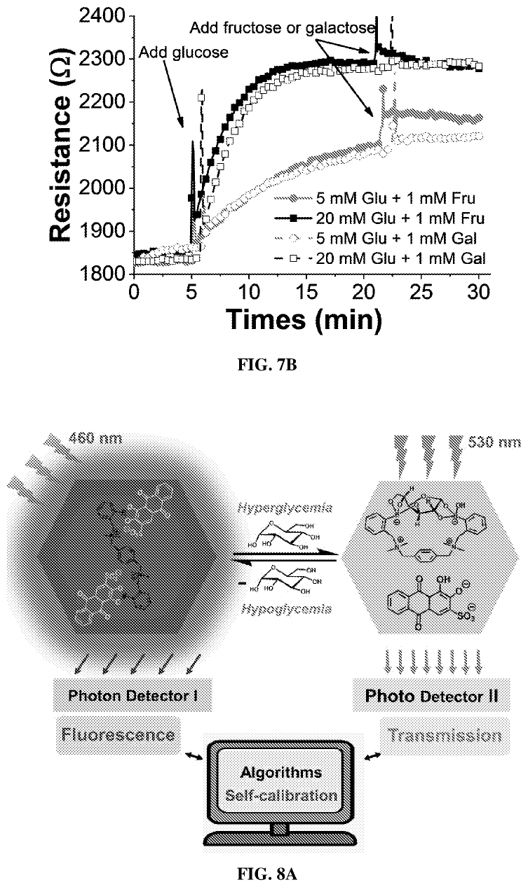

To assess reproducibility of the approach, quadruplicate measurements of R and conductance (a) were carried out at room temperature. Examination of the plots of percentage change (R or a) vs. glucose concentration ([glucose]) (FIG. 7A) reveals good agreement between measurements, demonstrating the accuracy and stability of the glucose sensing platform. Some of the statistical variations arise from changes in either solution volume or solution temperature (1-2% total conductance change per ° C.).

### Example 5. DBA2+ Based Conductive Assay Shows Negligible Effect on the Signal

Materials and Methods

Interference effects from other sugars, i.e. fructose, galactose, maltose, and lactose, were examined. Tests were performed at two different glucose concentrations typically experienced in diabetes, 5 mM (i.e. a physiological concentration of glucose) and 20 mM (i.e. a pathophysiological concentration of glucose) in 1 mL of testing solution. In these tests, interferent concentrations were higher or equal to 2.5 times the maximum plasma concentration (MPC). To the testing solution containing glucose, 2 μL or 5 μL of one of the interference solutions (containing fructose, galactose, maltose, or lactose) were added and the solution resistance (R) was monitored. The interference by 1 mM fructose or galactose was tested due to the considerable high affinity compared to the other disaccharides.

Results

Table 1 summarizes the results of Example 5.

TABLE 1
Effect of Interference Sugars on Performance of
Conductimetric Sensor.

| | Max | | | |
|---|---|---|---|---|
| | plasma | Interference | | Resistance |
| Interference | [sugar] | [sugar] | [Glucose] | increase |
| Sugar | (mM) | (mM) | (mM) | (%)^{a} |
| Fructose | 0.133 | 1 | 5 | 2.9 |
| | | | 20 | −0.3 |
| Galactose | 0.28 | 1 | 5 | 1.4 |
| | | | 20 | 0.6 |
| Maltose | 3.5 | 10 | 5 | 0.4 |
| | | | 20 | 1.6 |
| Lactose | 0.015 | 1 | 5 | 0.2 |
| | | | 20 | 1.6 |

^{a}Resistance increase is based on the resistance value of testing solution with 5 mM glucose or 20 mM glucose.

As shown in FIG. 7B, the addition of galactose had a negligible effect on solution resistance. The addition of fructose caused a 3% increase in resistance under low glucose (5 mM) conditions and only a transient increase under high glucose (20 mM) conditions. 10 mM maltose and 1 mM lactose showed no significant change to R.

### Example 6. DBA2+ Based Optical Sensor Shows Response to Glucose with Self-Calibration and Self-Correction Capability

Materials and Methods

The optical sensing system includes a diboronic acid molecule (DBA2+) that is selective to glucose and a dye (e.g. chromophore alizarin red S (ARS)). ARS can reversibly bind with DBA2+ at a 2:1 ratio, and offers two types of optical signals to probe glucose concentration (i.e. absorbance and fluorescence). An exemplary dual-model glucose sensing strategy is illustrated in FIG. 8A.

Standard glucose solutions were tested on C1 (100 μM ARS and 75 μM DBA2+), C2 (80 μM ARS and 75 μM DBA2+) and C3 (100 μM ARS and 60 μM DBA2+) solutions. The absorption and fluorescence spectra were measured on microplate reader. Their absorbance values were plotted vs. corresponding fluorescence intensity values (see FIG. 10A). The calibration curves for C1 (dotted line), C2 (straight line) and C3 (dashed line) were simulated with a fourth-degree polynomial method.

Two glucose samples were also tested randomly in one of the three ARS/DBA2+ solutions. The results were plotted to find the closest fitting curves as well as the current ARS/DBA2+ conditions.

Standard glucose concentrations were plotted as functions of the absorbance at 530 nm (dotted line) and the fluorescence intensity (straight line) at 600 nm from data of C2. Calibration curves were obtained with exponential fitting methods and used to determine glucose concentration.

Results

The recovery of the nature of ARS by glucose was observed. With the increase of glucose concentration, the absorption spectra decrease at the wavelength from 390 nm to 478 nm and increase at above 478 nm (FIG. 8B), and meanwhile the fluorescence intensity decrease to around one third of the original value, i.e. before adding glucose (FIG. 8C).

The absorbance and fluorescence signals are closely correlated to the total concentrations of both DBA2+ and ARS. Any changes on the concentration of DBA2+ or ARS change the calibration curves, which can be obtained through simulation or experiment. As shown in FIG. 10A, different DBA2+ and ARS compositions generate different curves by fitting the experimental absorbance and fluorescence values of ARS at several glucose levels.

Two unknown glucose sample measurement results were also plotted in FIG. 10A. The closest fitting curve can be identified in a qualitative way, which points out the composition of DBA2+ and ARS. The two calibration curves (glucose concentration vs. absorbance and fluorescence) for this composition were then used to calculate the glucose levels (FIG. 10B). Table 2 summarizes the calculated results. Without external calibration, this method can identify the right calibration curve to give accurate results through self-calibration and self-correction.

TABLE 2
Concentrations of two glucose samples determined from calibration curves for absorbance and fluorescence in
FIGS. 10A and 10B and from a standard method using a YSI 2900 instrument as comparison.

| | | | Calculated with fitting | | | Calculated with fitting | | | Calculated with fitting | | | | |
|---|---|---|---|---|---|---|---|---|---|---|---|---|---|
| | | | curves from C1 | | | curves from C2 | | | curves from C3 | | | Calculated with | |
| Test | Results | | | | Average | | | Average | | | Average | YSI 9000 | |
| No. | ABS | FL | [G_{A}] | [G_{F}] | (Error %) | [G_{A}] | [G_{F}] | (Error %) | [G_{A}] | [G_{F}] | (Error %) | [G_{Y}] | Error |
| 1-1 | 0.100 | 17.9 | 4.68 | 290 | 147 | 25.7 | 18.6 | 22.2 | 2.06 | 26.0 | 14.0 | 22.3 ± 1.2 | 0.44% |
| | | | | | (137%) | | | (22.7%) | | | (121%) | | |
| 1-2 | 0.0809 | 21.1 | −0.117 | 9.78 | 4.83 | 5.29 | 5.95 | 5.62 | −0.66 | 3.89 | 1.62 | 5.62 ± 0.31 | 0 |
| | | | | | (145%) | | | (8.30%) | | | (199%) | | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A diboronic acid compound having a structure of Formula I: 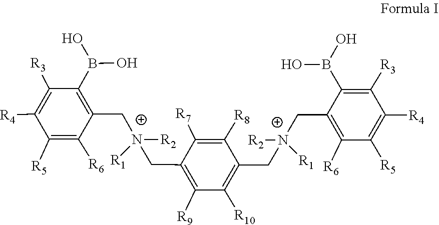
wherein R₁ and R₂ are independently an unsubstituted alkyl group, a substituted alkyl group, an unsubstituted heteroalkyl group, or a substituted heteroalkyl group; and
wherein R₃-R₁₀ are independently
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,
an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonate ester group containing one substituent, an ether group containing one substituent, an aminooxy group optionally containing one or two substituents at the amino nitrogen, or a hydroxyamino group optionally containing one or two substituents,
wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

2. The compound of claim 1, wherein R₁ and R₂ are independently unsubstituted or substituted alkyl groups, preferably unsubstituted or substituted C₁-C₁₀ alkyl groups, more preferably unsubstituted or substituted linear C₁-C₁₀ alkyl groups, most preferably unsubstituted or substituted methyl groups having a structure of Formula II: 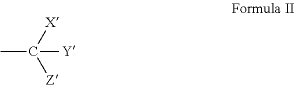
wherein X′, Y′, and Z′ are independently
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,
an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonate ester group containing one substituent, an ether group containing one substituent, an aminooxy group optionally containing one or two substituents at the amino nitrogen, or a hydroxyamino group optionally containing one or two substituents,
wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

3. The compound of claim 2, wherein X′, Y′, and Z′ are independently a hydrogen, a halogen atom, a nitrile group, a methyl group, or an unsubstituted aryl group.

4. The compound of claim 1, having a structure of Formula III:

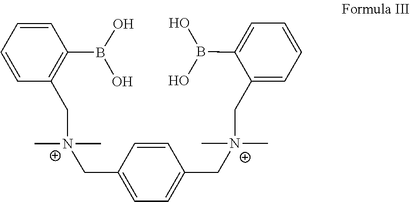 [SMILES: C[N+](C)(Cc1ccc(C[N+](C)(C)Cc2ccccc2B(O)O)cc1)Cc1ccccc1B(O)O]

5. A diboronic acid compound having a structure of Formula IV: 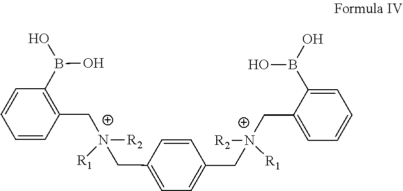
wherein R₁ and R₂ are independently an unsubstituted alkyl group, a substituted alkyl group, an unsubstituted heteroalkyl group, or a substituted heteroalkyl group, preferably an unsubstituted alkyl group or a substituted alkyl group, more preferably an unsubstituted C₁-C₁₀ alkyl group or a substituted C₁-C₁₀ alkyl group.

6. The compound of claim 1, further comprising counter ions to the tertiary amine groups.

7. The compound of claim 6, wherein the counter ions are halide anions, phosphate ion, hydrogen phosphate ion, dihydrogen phosphate ion, trihydrogen phosphate ion, or bicarbonate, or a combination thereof.

8.-9. (canceled)

10. The compound of claim 1, wherein the compound binds glucose with a K_{d} value between about 0.1 mM and about 30 mM.

11. The compound of claim 1, wherein the compound binds glucose with a K_{d} value at least about 2-times lower, at least about 5-times lower, at least about 10-times lower, at least about 15-times lower, or at least about 20-times lower than a K_{d} value for an interference sugar under the same conditions.

12. (canceled)

13. The compound of claim 1 having a pKa value between about 7.4 and about 10.5, preferably between about 8.5 and about 10.5, more preferably between about 9 and about 10.

14. (canceled)

15. The compound of claim 13, wherein the pKa value increases or decreases by about 1 unit, about 2 units, preferably about 3 units, more preferably about 4 units upon binding with glucose.

16. (canceled)

17. A conductivity sensor for measuring glucose concentration in a biological sample comprising
a reservoir wherein the compound claim 1 and a buffer solution are located therein;
a pair of electrodes; and
a membrane,
wherein the electrodes are in electrical communication with each other,
wherein an electrically conductive surface of each electrode is in contact with the buffer solution, and
wherein the membrane is configured to prevent or reduce ion exchange between the buffer solution and the biological sample.

18. A conductivity sensor for measuring glucose concentration in a biological sample comprising
a reservoir wherein the compound of claim 1 and buffer salts are located therein;
a pair of electrodes; and
a membrane,
wherein the electrodes are in electrical communication with each other,
wherein the compound and the buffer salts are in the form of a solid, optionally in the form of a powder.

19. The conductivity sensor of claim 17, wherein the reservoir is defined by side walls and a bottom surface, and contains an opening configured to allow the biological sample to enter the reservoir, optionally wherein an electrically conductive surface of each electrode is part of or forms one or more of the side walls and/or bottom surface of the reservoir.

20. The conductivity sensor of claim 17, wherein the membrane is located adjacent to the opening of the reservoir, and defines an outer surface that encloses the buffer solution or solid buffer salts and compound inside of the reservoir.

21. The conductivity sensor of claim 17, wherein the membrane is a bipolar membrane.

22.-27. (canceled)

28. An optical sensor comprising the compound claim 1, a dye, a light source, and a detector
wherein the compound and the dye form a complex (DBA-D complex).

29. The optical sensor of claim 28, further comprising a processor, a transmitter, or an output display, or a combination thereof.

30.-33. (canceled)

34. A continuous glucose monitoring system (CGMS) comprising:
(a) (i) a conductivity sensor comprising
a reservoir wherein the compound of claim 1 and buffer salts or a buffer solution are located therein;
a pair of electrodes; and
a membrane,
wherein the electrodes are in electrical communication with each other,
wherein the reservoir is defined by side walls and a bottom surface, and contains an opening configured to allow the biological sample to enter the reservoir, optionally wherein an electrically conductive surface of each electrode is part of or forms one or more of the side walls and/or bottom surface of the reservoir, optionally wherein the membrane is located adjacent to the opening of the reservoir, and defines an outer surface that encloses the buffer solution or solid buffer salts and compound inside of the reservoir,
or (ii) an optical sensor comprising the compound of claim 1, a dye, a light source, and a detector, wherein the compound and the dye form a complex (DBA-D complex), optionally further comprising a processor, a transmitter, or an output display, or a combination thereof; and
optionally further comprising
(b) a bipolar membrane; and/or
(c) a microneedle, optionally an array of microneedles for fluid extraction.

35. The continuous glucose monitoring system of claim 34 comprising two or more of the conductivity sensors or two or more of the optical sensors.

36.-37. (canceled)
